(19) United States
(12) Patent Application Publication
(10) Pub. No.: US 2024/0309068 A1
(43) Pub. Date: Sep. 19, 2024

(54) RECOMBINANT ADENO-ASSOCIATED VIRAL VECTORS FOR TREATING BIETTI CRYSTALLINE DYSTROPHY

(71) Applicant: SHANGHAI VITALGEN BIOPHARMA CO., LTD., Shanghai (CN)

(72) Inventors: Lu GUO, Haidian (CN); Yezheng TAO, Shanghai (CN); Shin-Shay TIAN, Shanghai (CN); Bin QU, Shanghai (CN); Wei LI, Beijing (CN); Xi ZHU, Shanghai (CN); Xiaoping ZHAO, Shanghai (CN)

(73) Assignee: SHANGHAI VITALGEN BIOPHARMA CO., LTD., Shanghai (CN)

(21) Appl. No.: 18/410,043

(22) Filed: Jan. 11, 2024

Related U.S. Application Data
(63) Continuation of application No. PCT/CN2022/106089, filed on Jul. 15, 2022.

(30) Foreign Application Priority Data
Jul. 15, 2021 (WO) PCT/CN2021/106619

(51) Int. Cl. C07K 14/80; A61K 38/41; A61K 48/00; C12N 15/86

(52) U.S. Cl. CPC C07K 14/80; A61K 38/415; A61K 48/0066; C12N 15/86; C12N 2750/14143; C12N 2830/48; C12N 2830/50

(57) ABSTRACT

The present disclosure provides a recombinant adeno-associated vector comprising a codon-optimized sequence encoding CYP4V2 linked to selected gene expression regulatory sequences and its use in treating Bietti Crystalline Dystrophy (BCD).

### FIELD OF THE INVENTION

The present disclosure provides an expression cassette and recombinant adeno-associated virus (rAAV) vector comprising a nucleic acid encoding CYP4V2. Also provided are viral particles comprising a rAAV vector, a composition comprising the viral particles, and uses thereof.

### BACKGROUND

Recent progresses have demonstrated that gene therapy holds great promises in treating human genetic disorders[1]. Over 6,000 of inherited diseases have been described[2], the number of which is still increasing with further disclosure of genome sequencing techniques[3]. The discovery of safer delivering vectors and the revelation of new gene editing techniques in recent years significantly enhanced the toolbox for treating these genetic diseases, with which the deficient genes could either be supplemented by exogenetic DNAs that carried by a therapeutic vector or be corrected in situ permanently by gene editing[4]. Contrary to this promising prospect, the list of genetic disorders that are clinically treatable grows slowly[5]. A major limiting factor to this situation is the lack of human translatable animal models, which impeded the therapeutic effect evaluation of gene therapies[6].

Bietti Crystalline Dystrophy (BCD, MIM 210370) is an autosomal recessive inherited disease first described by the Italian ophthalmologist G. B. Bietti in 1937, which was named for its distinguishing yellow-white crystalline deposits observed in patient's fundus[7]. BCD is accounted for ˜3% of all nonsyndromic retinitis pigmentosa (RP) and for ˜10% of nonsyndromic autosomal recessive RP in Europe[8]. BCD also appears to be more common in East Asia, especially in China, where the genetic mutation frequency is estimated to be 1 in 20,000[9].

In 2000, it was identified that the genetic basis of BCD is associated with chromosome 4q35[8]. CYP4V2, a member of the cytochrome P450 superfamily, was identified as the disease-causing gene of BCD in 2004[10]. Besides the crystalline deposits in retina and cornea, altered fatty acid concentration was also found in the serum of BCD patients, which implied a dysregulation of lipid metabolism[11,12]. More importantly, BCD patients inevitably develop vision loss and night blindness between 20 and 40 years of age and eventually progress to legal blindness between the ages of 50 and 60[11]. Unfortunately, 80 years after the first discovery of BCD, there is still no treatment available for this severe blinding disease.

AAV has become a preferred delivery tool for gene therapies for treating genetic disorders such as BCD due to its demonstrated long-term transgene expression, selective tropism and non-pathogenic nature.

CN111733174B discloses a construct which can be packaged into AAV vectors and comprises both coding sequences of CYP4V2 and RdCVF for treating BCD. The construct comprises a CAG promoter and a BGH polyadenylation signal site.

CN109136266A also provides construct for expressing CYP4V2 by rAAV. In the packaging plasmid, a sequence encoding short peptide C9 is inserted to enable a specific binding to CD59 antigen on human RPE cell membrane. In addition, a sequence encoding HRH peptide is inserted along with the coding sequence of CYP4V2 to inhibit VEGF.

Both CN111733174B and CN109136266A use the wild type coding sequence of CYP4V2.

Accordingly, there is a need in the art for improved gene therapy compositions and methods that can efficiently and safely restore CYP4V2 gene function in BCD patients.

### SUMMARY OF THE INVENTION

The present inventors have developed rAAV vectors comprising codon-optimized coding sequence of CYP4V2 and certain combinations of gene expression regulatory sequences, which achieved significantly enhanced expression levels (˜26.1 times) as compared to the previously published form[13], thus completing the invention.

Therefore, in a first aspect, the present disclosure relates to an isolated nucleic acid molecule, comprising a nucleotide sequence selected from a group consisting of SEQ ID NOs: 2-17 and encoding human CYP4V2. In a more preferred embodiment, the nucleotide sequence is selected from SEQ ID NOs: 8, 9, 15, 16 and 17. In an even more preferred embodiment, the nucleotide sequence is SEQ ID NO: 8 or SEQ ID NO: 16. In the most preferred embodiment, the nucleotide sequence is SEQ ID NO: 16.

In an embodiment of the first aspect, the isolated nucleic acid molecule further comprises a promoter which is operatively linked to the 5′ of the nucleotide sequence encoding CYP4V2. Preferably, the promoter is CAG promoter of SEQ ID NO: 35.

In one embodiment, the isolated nucleic acid molecule further comprises a polyadenylation sequence at the 3′ of the nucleotide sequence encoding CYP4V2 polypeptide.

Preferably, the polyadenylation sequence is bovine growth hormone (bGH) polyA, synthesized polyA (SPA) or Simian Virus 40 (SV40) polyA, more preferably SV40 polyA.

In another embodiment, the isolated nucleic acid further comprises a Woodchuck Hepatitis Virus posttranscriptional regulatory element (WPRE) located between the CYP4V2 coding sequence and the SV40 polyA sequence.

In one preferred embodiment, the isolated nucleic acid molecule comprises
(a) a nucleotide sequence of any one of SEQ ID NOs: 18-34, or
(b) a nucleotide sequence having at least 85% homology, at least 90% homology, at least 95% homology, at least 96% homology, at least 97% homology, at least 98% homology, at least 99% homology to any one of SEQ ID NO: 18-34.

In a second aspect, the present disclosure provides a recombinant AAV (rAAV) vector comprising the nucleic acid molecule of the first aspect.

In one embodiment, the rAAV vector comprises at least one ITR, preferably two ITRs. In a preferred embodiment, the two ITRs are derived from AAV2 ITR.

In a third aspect, the present disclosure provides a viral particle comprising the recombinant AAV vector of the second aspect and AAV capsid selected from AAV1, AAV2, AAV4, AAV5, AAV7, AAV8, AAV9, AAVrh10, AAV2.7m8, AAVAnc80L65 and the variants thereof. Preferably, the capsid is AAV8.

In a fourth aspect, the present disclosure provides a pharmaceutical composition comprising the viral particle of the third aspect and a pharmaceutically acceptable excipient.

In a fifth aspect, the present disclosure provides use of the rAAV vector in the manufacture of a medicament for treating or preventing Bietti Crystalline Dystrophy (BCD) or any other diseases of retinal pigment epithelium (RPE) atrophy.

### BRIEF DESCRIPTION OF THE DRAWINGS

FIG. 1 is an illustrative diagram showing the constructs CMV-hCyp4v2 (upper panel) and CAG-hCyp4v2 (BCD1, lower panel).

FIG. 2 is a histogram showing the CYP4V2 transgene protein expression levels of the construct CMV-hCyp4v2 in comparion with the construct CAG-hCyp4v2 (BCD1) in HEK293.

FIG. 3 shows the evaluation of the CYP4V2 transgene protein expression levels of the constructs BCD1-BCD17 in HEK293 cells.

FIG. 4 shows the evaluation of the CYP4V2 transgene protein expression levels of the constructs BCD1-BCD17 in ARPE-19 cells.

FIG. 5 shows the CYP4V2 transgene protein expression levels of the constructs BCD8, BCD9, BCD15, BCD16, and BCD17 in HEK293 cells. (A) Western blot. (B) Histograms.

FIG. 6 shows the CYP4V2 transgene protein expression levels of the constructs BCD8, BCD9, BCD15, BCD16, and BCD17 in ARPE-19 cells. (A) Western blot. (B) Histograms.

FIG. 7 shows the CYP4V2 transgene protein expression levels of the packaged AAV8-BCD1, AAV8-BCD8, and AAV8-BCD16 in the transduced ARPE-19 cells. (A) Western blot. (B) Histograms.

### DETAILED DESCRIPTION OF THE INVENTION

Unless specifically defined elsewhere in this document, all the technical and scientific terms used herein have the meaning commonly understood by one of ordinary skill in the art to which this invention belongs.

As used herein, including the appended claims, the singular forms of words such as “a”, “an”, and “the”, include their corresponding plural references unless the context clearly dictates otherwise.

In the context of the present disclosure, unless being otherwise indicated, the wording “comprise”, and variations thereof such as “comprises” and “comprising” will be understood to imply the inclusion of a stated element, e.g. an amino acid sequence, a nucleotide sequence, a property, a step or a group thereof, but not the exclusion of any other elements, e.g. amino acid sequences, nucleotide sequences, properties and steps. When used herein the term “comprise” or any variation thereof can be substituted with the term “contain”, “include” or sometimes “have” or equivalent variation thereof. In certain embodiments, the wording “comprise” also include the scenario of “consisting of”.

The abbreviation “CYP4V2” refers to cytochrome P450 family 4 subfamily V member 2, which is a gene coding for CYP4V2 protein of the cytochrome P450 hemethiolate protein superfamily involved in oxidizing various substrates in the metabolic pathway. Defects in the gene CYP4V2 are known to cause BCD and also fundus dystrophy. The CYP4V2 mentioned here refers to human CYP4V2 in the context of the present disclosure unless otherwise indicated.

### Isolated Nucleic Acid Encoding CYP4V2 Protein.

The present disclosure provides an isolated nucleic acid sequence comprising a nucleotide sequence coding for CYP4V2 protein, specifically human CYP4V2. By “isolated nucleic acid”, it means a DNA or RNA which is removed from all or a portion of a polynucleotide in which the isolated polynucleotide is found in nature, or is linked to a polynucleotide to which it is not linked in nature. An isolated nucleic acid molecule “comprising” a specific nucleotide sequence may include, in addition to the specified sequence, operably linked regulatory sequences that control expression of the coding region of the recited nucleic acid sequences. Due to the codon degeneracy, one skilled in the art can understand that a specific amino acid sequence can be coded by different nucleotide sequences.

The nucleotide sequence coding for CYP4V2 of the present disclosure has been subjected to codon optimization and screening, resulting in an enhanced expression level as compared to the wild-type coding sequence without codon optimization.

For example, the codon optimized coding sequence of CYP4V2 can achieve an expression level about 2-fold, about 3-fold, about 4-fold, about 5-fold, about 10-fold, about 15-fold, or about 20-fold as compared to the wild-type coding sequence by Western blot analysis of CYP4V2 protein expression levels in HEK293 cells and ARPE-19 cells.

Preferably, the codon optimized coding sequence of CYP4V2 has a reduced CpG number and/or a reduced number of CpG island. For example, the codon optimized coding sequence comprises less than 100 CpG, less than 70 CpG, less than 20 CpG, less than 15 CpG, less than 10 CpG, less than 5 CpG or 0 CpG. For example, the codon optimized coding sequence does not have CpG islands. By “CpG island” it stands for a DNA region of at least 200 bp in length, a GC percentage content greater than 50%, and an observed-to-expected CpG ratio greater than 60%.

For example, the codon-optimized coding sequence has a percentage sequence identity of lower than 80% as compared to the wild-type coding sequence of CYP4V2 as shown in SEQ ID NO: 1. The percentage identity of two sequences can be calculated by known programs in the art. Due to the different parameters for alignment and different definition of identity, the calculated percentage identity of two sequences may vary depending on the program being used to do the calculation. In the present disclosure, blastn is used to obtain the percentage identity of two nucleotide sequences, and specifically a “highly similar sequences (megablast)” mode is selected.

The codon-optimized coding sequence can be selected from a group consisting of the nucleotide sequences as set forth in SEQ ID NOs: 2-17. Preferably, the codon-optimized coding sequence is any one of SEQ ID NOs: 8, 9, 15, 16 and 17. More preferably, the codon-optimized coding sequence is SEQ ID NO: 16.

The regulatory sequences comprised in the nucleic acid molecule can be selected from one or more of promoter, enhancer, polyadenylation sequence, and translation termination signal. A certain combination of regulatory sequences of the present disclosure can achieve unexpected effect in improving the expression efficiency of the coding sequence.

The promoter of the present disclosure can be a constitutive promoter, a tissue-specific promoter or a cell type-specific promoter. For example, the promoter can be an RPE cell-specific promoter. The promoter can be the native promoter of CYP4V2. In a more preferred embodiment, the promoter is a CAG promoter having a sequence as set forth in SEQ ID NO. 35. CAG promoter is a strong constitutive promoter which drives high levels of gene expression in mammalian expression vectors. CAG promoter consists of a cytomegalovirus (CMV) early enhancer element, a promoter which is picked from the first exon and the first intron of chicken beta-actin gene and a splice acceptor of the rabbit beta-globin gene.

The Kozak consensus sequence (Kozak sequence), named after the scientist who discovered it, is a nucleic acid motif in most eukaryotic mRNA transcripts that naturally functions as the protein translation initiation site[14]. Kozak sequence ensures the protein is correctly translated and enhances protein expression.

In addition, the nucleic acid of the present disclosure can further comprise an intron inserted between the promoter and the coding sequence. As known in the art, some introns may enhance the expression of genes in eukaryotes. The intron of the present disclosure can be a part of the naturally occurring intron of the CYP4V2 gene.

The polyadenylation sequence of the present disclosure can be bGH polyA, SPA or SV40 polyA [15], preferably SV40 polyA. Anyone of the polyA can be combined with a Woodchuck Hepatitis Virus posttranscriptional regulatory element (WPRE)[16], which is a DNA sequence, when transcribed, creates a tertiary structure enhancing expression, and configured as WPRE-bGH polyA, WPRE-SPA or WPRE-SV40 poly A, respectively.

In preferred embodiment of the present disclosure, the nucleic acid sequence comprises a nucleotide sequence encoding for a CAG promoter, a Kozak sequence, a codon-optimized coding sequence of CYP4V2 gene, and a WPRE-SV40 polyA. For example, the isolated nucleic acid sequence comprises a nucleotide sequence of any one of SEQ ID NOs: 18-34. In a more preferred embodiment, the isolated nucleic acid sequence comprises a nucleotide sequence of any one of SEQ ID NOs: 25, 26, 32, 33, and 34. In the most preferred embodiment, the isolated nucleic acid sequence comprises a nucleotide sequence of SEQ ID NO: 33.

rAAV Vectors and Viral Particles

The nucleic acid molecule of the present disclosure can be constructed into a recombinant AAV vector, to obtain rAAV particles for delivery into treatment subjects.

In addition to the inserted nucleotide sequence as described above, the rAAV vectors are in single stranded form. The rAAV vector is usually comprised of two inverted terminal repeat (ITR) sequences at both ends of the inserted nucleotide sequence. The ITR of the present disclosure can be ITR derived from any AAV serotypes. When reference is made to serotype of AAV ITR, the phrase “derived from” means that the ITR can be the ITR of a certain serotype or a variant derived therefrom with modification(s). In a preferred embodiment of the present disclosure, the rAAV vector comprises two ITRs derived from AAV2[17]. For example, the rAAV vector comprises two AAV2 ITRs, or comprises a wild-type AAV2 ITR and an AAV2 ITR variant lacking region C or region C′. For example, the wild-type AAV2 ITR locates at a position at 5′ of the inserted nucleotide sequence, while the AAV2 ITR variant locates at a position at 3′ of the inserted nucleotide sequence; or vice versa.

The rAAV genome was packaged into an AAV capsid. The capsid can be derived from any AAV serotype known in the art or characterized in the future. The capsid and ITRs can be derived from the same serotype of AAV or from different serotypes of AAV. Preferably, the capsid is suitable for eye delivery, e.g., subretinal, intravitreal or intraocular delivery. In a specific embodiment, the AAV vector comprises a capsid of AAV1, AAV2, AAV4, AAV5, AAV7, AAV8, AAV9, AAVrh10, AAV2.7m8, or AAVAnc80L65 serotype, or a variant thereof.

In a preferred embodiment of the present disclosure, the rAAV is comprised of AAV serotype 8 capsids[18]. Evidence have shown that ssAAV8 transduces photoreceptors and retinal pigment epithelial cells (RPE) more efficiently than ssAAV2 and ssAAV5. Under the guidance of the capsid proteins, the virus transduces target cells and then the transferred genome containing CYP4V2 gene and regulatory elements is released into the targeted RPE. The released rAAV genome remains stable and independent from the host genome to stably produce functional CYP4V2 protein in the treatment subjects.

### Pharmaceutical Composition

The term “pharmaceutical composition” refers to a composition suitable for delivering to a subject. The pharmaceutical composition of the present disclosure comprises the isolated nucleic acid, the rAAV vector or the viral particle of the present disclosure and a pharmaceutically acceptable excipient. Conventional pharmaceutically acceptable excipients are known in the art and can be solid or liquid excipients.

### Therapeutic Uses

The rAAV, viral particle or composition of the present disclosure can be used to treat or prevent BCD, which is related to CYP4V2 mutations. In addition, the rAAV, viral particle or composition of the present disclosure may also be effective in treating or preventing other conditions or diseases related to retinal pigment epithelium (RPE) atrophy, such as fundus dystrophy.

The term “treat”, “treating” or “treatment” includes cure or at least alleviate the symptoms of BCD, or conditions or diseases related to RPE atrophy.

### Administration

The terms “administration” and “administering” as used herein, when applied to a subject, e.g. an animal, including human, or to cells, tissue, organ, or biological fluid, mean contact of an exogenous pharmaceutical, therapeutic, diagnostic agent, or composition to the subject, cell, tissue, organ, or biological fluid. The term “administration” also includes in vitro and ex vivo treatments, e.g., of a cell, by a reagent, diagnostic, binding compound, or by another cell.

In the present disclosure, the viral particle or the pharmaceutical composition of the present application is preferably delivered through intra-ocular delivery, e.g., sub-retinal injection.

### EXAMPLES

### Example 1. Selection of Promoter

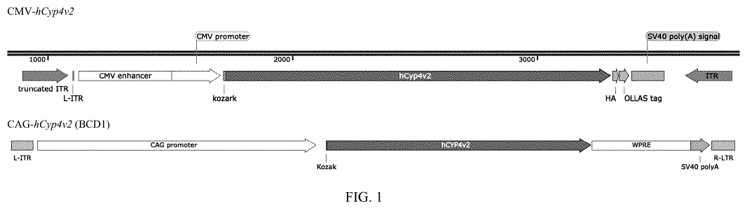

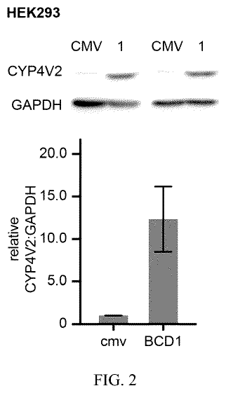

The inventors first prepared a CYP4V2-expression AAV vector comprised of two ITRs, a CMV promoter, a wild type CYP4V2 CDS and SV40 polyA[13]. Due to the reported silencing risk of CMV promoter[19], the CMV promoter was latere replaced with a CAG promoter, resulting in a new vector designated as “CAG-hCyp4v2 (BCD1)” which showed significantly improved CYP4V2 protein expression (FIG. 1, lower panel).

### Example 2. Codon Optimization Increased Cyp4v2 Expression

To obtain a transgene construct which could provide higher expression levels of the CYP4V2 protein, the coding sequence of CYP4V2 was codon optimized to improve codon usage frequency in human cells. In addition, the GC content and CpG islands of the modified coding sequence were reduced to minimize the potential for TLR9-mediated immune responses in vivo. Based on the above criteria, 16 different coding sequences (SEQ ID NOs: 2-17) were designed, synthesized and cloned into CAG promoter to obtain 16 more transgene constructs (BCD2-BCD17). Following Table 1 shows the CpG number, CpG island number of SEQ ID NOs: 2-17, as well as their sequence homology to the wild-type sequence.

TABLE 1
CpG content, CpG island number and sequence identity
percentages of 16 codon-optimized sequences in
comparison to the wild-type coding sequence

| Construct ID | Codon ID | CpG number | CpG islands | identity % |
|---|---|---|---|---|
| BCD1 | CYP4V2_wt | 50 | Yes | 100 |
| BCD2 | CYP4V2_co1 | 69 | Yes | 77.15 |
| BCD3 | CYP4V2_co2 | 63 | Yes | 78.23 |
| BCD4 | CYP4V2_co3 | 62 | Yes | 76.89 |
| BCD5 | CYP4V2_co4 | 65 | Yes | 77.34 |
| BCD6 | CYP4V2_co5 | 61 | Yes | 75.56 |
| BCD7 | CYP4V2_co6 | 0 | No | 79.25 |
| BCD8 | CYP4V2_co7 | 12 | No | 78.68 |
| BCD9 | CYP4V2_co8 | 0 | No | 79.12 |
| BCD10 | CYP4V2_co9 | 0 | No | 78.99 |
| BCD11 | CYP4V2_co10 | 0 | No | 79.69 |
| BCD12 | CYP4V2_co11 | 0 | No | 78.29 |
| BCD13 | CYP4V2_co12 | 0 | No | 78.8 |
| BCD14 | CYP4V2_co13 | 0 | No | 79.44 |
| BCD15 | CYP4V2_co14 | 0 | No | 78.74 |
| BCD16 | CYP4V2_co15 | 0 | No | 78.74 |
| BCD17 | CYP4V2_co16 | 1 | No | 76.89 |

### Example 3. Transfection of HEK293 and ARPE-19 Cells and Detection of Expression Level

HEK293 and ARPE-19 cells were maintained in DMEM+10% FBS and passaged every 3 days by TrypLE. The day before transfection, HEK293 cells were inoculated to a 24-well plate at a density of 1×10⁵ cells/cm², while ARPE-19 cells were inoculated to a 24-well plate at a density of 7×10⁴ cells/cm². Plasmids were transfected using Lipofectamine 3000 Transfection Reagent (Invitrogen, L3000008) following the user's guide. 72 hours after transfection, cells were collected in RIPA lysis buffer (Beyotime, P0013C) with a protease inhibitor cocktail (Roche, 04693159001) and SDS-PAGE loading buffer (Cowin Bio, CW0027), denatured for 15 min at 95° C., centrifuged at 12,000 rpm for 10 min. Supernatants were separated in 4%-10% SDS-PAGE gel (Cowin Bio, CW0022M), and blotted onto 0.45 μm NC transfer membrane (Merck, HATF00010).

The protein expression levels of CYP4V2 and the housekeeping gene GAPDH were detected by an antibody against the human CYP4V2 (Sigma, HPA029122) and GAPDH (Abcam, ab8245), respectively. The gray value of bands were calculated and normalized to BCD1.

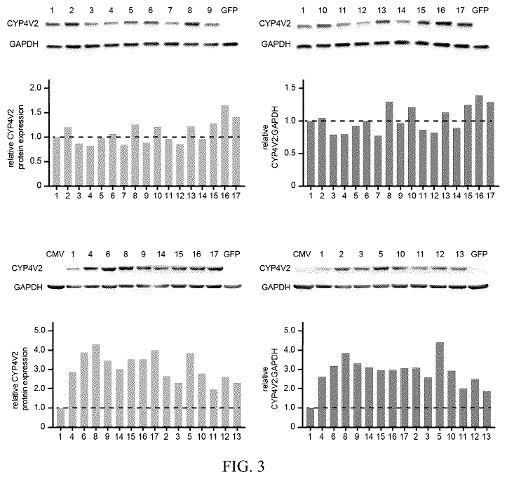

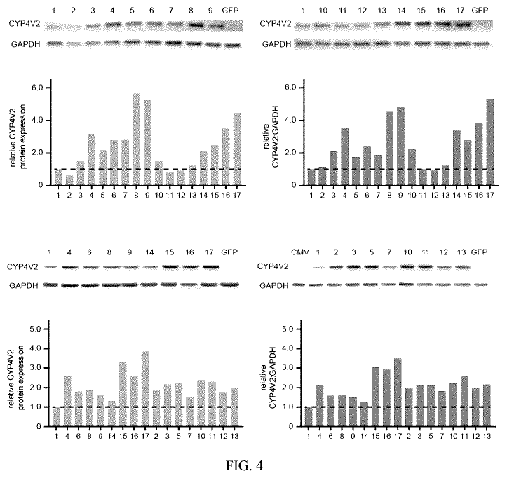

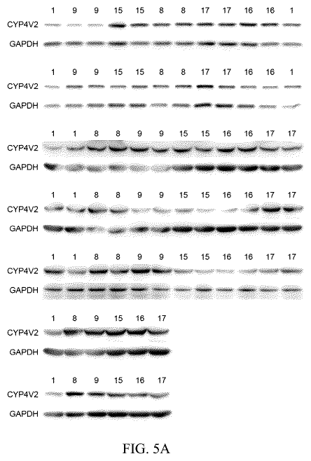

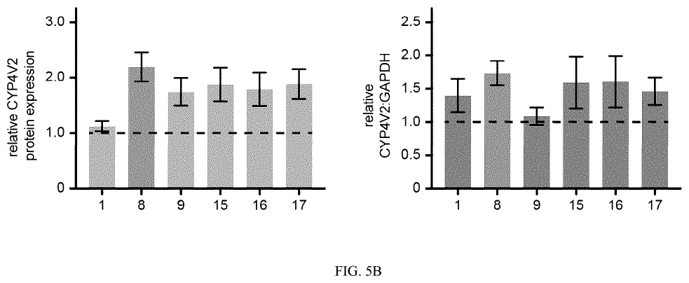

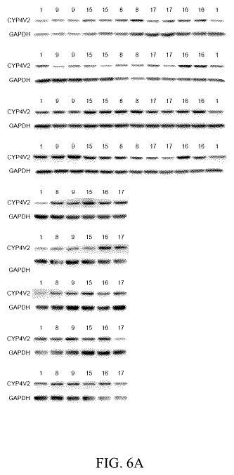

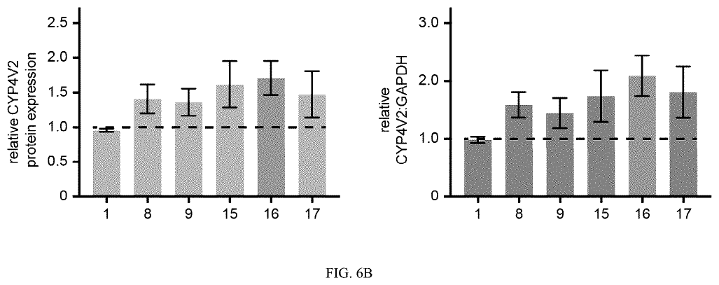

The results from repeated experiments showed that transgene constructs BCD8, BCD9, BCD15, BCD16, BCD17 expressed CYP4V2 protein at higher levels among the 16 tested codon optimized vectors as evaluated in HEK293 cells (FIG. 3) and ARPE-19 cells (FIG. 4). Accordingly, transgene constructs BCD8, BCD9, BCD15, BCD16 and BCD17 were selected for further evaluation, using BCD1 as a control. Western blot (WB) results are shown in FIG. 5A and FIG. 6A. The gray value of all the bands were calculated, analyzed and normalized to BCD1 (FIG. 5B and FIG. 6B).

Based on the Western blot results, it was determined that BCD8 exhibited the highest CYP4V2 protein expression levels in HEK293 cells (FIG. 5A and FIG. 5B) and BCD16 exhibited the highest CYP4V2 protein expression levels in ARPE-19 cells (FIG. 6A and FIG. 6B).

Later, BCD1, BCD8, and BCD16 were packaged to replication-defective AAV8, respectively. AAV8-BCD1, AAV8-BCD8 and AAV8-BCD16 virus particles were used to transduce ARPE-19 at MOI=5×10⁵ in the presence of 2 mM hydroxyurea.

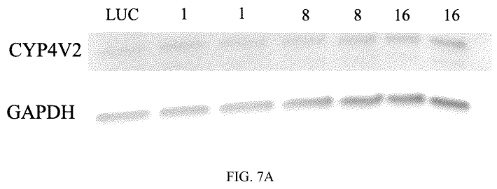

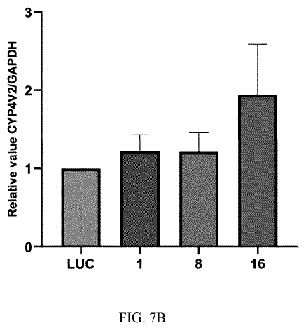

The AAV transduction experiment showed that AAV8-BCD16 had the highest expression levels (FIG. 7A and FIG. 7B).

### REFERENCES

1. Friedmann T, Roblin R. Gene therapy for human genetic disease? Science. 1972; 175: 949-955.
2. Mckusick V A. Mendelian inheritance in man and its online version, omim. Am J Hum Genet. 2007; 80: 588-604.
3. Chen R, Shi L S, Hakenberg J, Naughton B, Sklar P, Zhang J G et al. Analysis of 589,306 genomes identifies individuals resilient to severe mendelian childhood diseases. Nat Biotechnol. 2016; 34: 531-538.
4. Dunbar C E, High K A, Joung J K, Kohn D B, Ozawa K, Sadelain M. Gene therapy comes of age. Science. 2018; 359.
5. Ginn S L, Amaya A K, Alexander I E, Edelstein M, Abedi M R. Gene therapy clinical trials worldwide to 2017: An update. J Gene Med. 2018; 20.
6. Casal M, Haskins M. Large animal models and gene therapy. Eur J Hum Genet. 2006; 14: 266-272.
7. Bietti G B. Ueber familiaeres vorkommen von “retinitis Punctata albescens” (verbunden mit “dystrophia Marginalis cristallinea corneae”): Glitzern des glaskoerpers und anderen degenerativen augenveraenderungen. Klin Mbl Augenheilk. 1937; 99: 737-756.
8. Jiao X D, Munier F L, Iwata F, Hayakawa M, Kanai A, Lee J et al. Genetic linkage of bietti crystallin corneoretinal dystrophy to chromosome 4q35. Am J Hum Genet. 2000; 67: 1309-1313.
9. Hu D N. Ophthalmic genetics in china. Ophthalmic Paed Gen. 1983; 2: 39-45.
10. Li A, Jiao X, Munier F L, Schorderet D F, Yao W, Iwata F et al. Bietti crystalline corneoretinal dystrophy is caused by mutations in the novel gene cyp4v2. Am J Hum Genet. 2004; 74: 817-826.
11. Kaiser-Kupfer M I, Chan C-C, Markello T C, Crawford M A, Caruso R C, Csaky K G et al. Clinical biochemical and pathologic correlations in bietti's crystalline dystrophy. American Journal of Ophthalmology. 1994; 118: 569-582.
12. Lai T Y Y, Chu K O, Chan K P, Ng T K, Yam G H F, Lam DSC et al. Alterations in serum fatty acid concentrations and desaturase activities in bietti crystalline dystrophy unaffected by cyp4v2 genotypes. Invest Ophth Vis Sci. 2010; 51: 1092-1097.
13. Qu B, Wu S, Jiao G, Zou X, Li Z, Guo L et al. Treating bietti crystalline dystrophy in a high-fat diet-exacerbated murine model using gene therapy. Gene Therapy. 2020.
14. Kozak M. An analysis of 5′-noncoding sequences from 699 vertebrate messenger rnas. Nucleic acids research. 1987; 15: 8125-8148.
15. Connelly S, Manley J L. A functional mrna polyadenylation signal is required for transcription termination by rna polymerase ii. Genes & development. 1988; 2: 440-452.
16. Zufferey R, Donello J E, Trono D, Hope T J. Woodchuck hepatitis virus posttranscriptional regulatory element enhances expression of transgenes delivered by retroviral vectors. Journal of virology. 1999; 73: 2886-2892.
17. Grimm D, Kern A, Rittner K, Kleinschmidt J A. Novel tools for production and purification of recombinant adenoassociated virus vectors. Hum Gene Ther. 1998; 9: 2745-2760.
18. Gao G P, Alvira M R, Wang L, Calcedo R, Johnston J, Wilson J M. Novel adeno-associated viruses from rhesus monkeys as vectors for human gene therapy. Proceedings of the National Academy of Sciences of the United States of America. 2002; 99: 11854-11859.
19. Tenenbaum L, Chtarto A, Lehtonen E, Velu T, Brotchi J, Levivier M. Recombinant aav—mediated gene delivery to the central nervous system. The Journal of Gene Medicine: A cross—disciplinary journal for research on the science of gene transfer and its clinical applications. 2004; 6: S212-S222.

1. An isolated nucleic acid molecule, comprising a nucleotide sequence which is at least one of SEQ ID NOs: 2-17 and that encodes human CYP4V2 polypeptide.

2. The isolated nucleic acid molecule of claim 1, wherein the nucleotide sequence is SEQ ID NOs: 8, 9, 15, 16 or 17.

3. The isolated nucleic acid molecule of claim 2, wherein the nucleotide sequence is SEQ ID NO: 8 or SEQ ID NO: 16.

4. The isolated nucleic acid molecule of claim 1, further comprising a promoter operatively linked to the 5′ of the nucleotide sequence encoding CYP4V2.

5. The isolated nucleic acid molecule of claim 4, wherein the promoter is a CAG promoter.

6. The isolated nucleic acid molecule of claim 1, further comprising a polyadenylation sequence at the 3′ of the nucleotide sequence encoding CYP4V2.

7. The isolated nucleic acid molecule of claim 6, wherein the polyadenylation (polyA) sequence is bovine growth hormone (bGH) polyA, synthesized polyA (SPA) or Simian Virus 40 (SV40) polyA.

8. The isolated nucleic acid molecule of claim 1, further comprising a Woodchuck Hepatitis Virus posttranscriptional regulatory element (WPRE).

9. The isolated nucleic acid molecule of claim 1, comprising any of (a) or (b)
(a) a nucleotide sequence of any one of SEQ ID NOs: 18-34, or
(b) a nucleotide sequence having at least 85% homology, at least 90% homology to any one of SEQ ID NO: 18-34.

10. A recombinant AAV vector, comprising the nucleic acid molecule of claim 1.

11. The recombinant AAV vector of claim 10, comprising one or two inverted terminal repeats.

12. The recombinant AAV vector of claim 11, comprising two AAV2 inverted terminal repeats.

13. A viral particle comprising the recombinant AAV vector of claim 10 packaged into an AAV capsid.

14. The viral particle of claim 13, wherein the AAV capsid is AAV8 capsid.

15. A pharmaceutical composition comprising the viral particle of claim 13, and a pharmaceutically acceptable excipient.

16. A method of treating or preventing Bietti Crystalline Dystrophy (BCD) comprising administering the rAAV vector of claim 10 to a subject in need thereof.


SEQUENCE LISTING (36 sequences)

SEQ ID NO: 1 - DNA, 1578 bp, Homo sapiens
  source 1..1578: mol_type = genomic DNA
atggcggggctctggctggggctcgtgtggcagaagctgctgctgtggggcgcggcgagtgccctttccctggccggcgccagtctggtcctgagcctgctgcagagggtggcgagctacgcgcggaaatggcagcagatgcggcccatccccacggtggcccgcgcctacccactggtgggccacgcgctgctgatgaagccggacgggcgagaattttttcagcagatcattgagtacacagaggaataccgccacatgccgctgctgaagctctgggtcgggccagtgcccatggtggccctttataatgcagaaaatgtggaggtaattttaactagttcaaagcaaattgacaaatcctctatgtacaagtttttagaaccatggcttggcctaggacttcttacaagtactggaaacaaatggcgctccaggagaaagatgttaacacccactttccattttaccattctggaagatttcttagatatcatgaatgaacaagcaaatatattggttaagaaacttgaaaaacacattaaccaagaagcatttaactgctttttttacatcactctttgtgccttagatatcatctgtgaaacagctatggggaagaatattggtgctcaaagtaatgatgattccgagtatgtccgtgcagtttatagaatgagtgagatgatatttcgaagaataaagatgccctggctttggcttgatctctggtaccttatgtttaaagaaggatgggaacacaaaaagagccttcagatcctacatacttttaccaacagtgtcatcgctgaacgggccaatgaaatgaacgccaatgaagactgtagaggtgatggcaggggctctgccccctccaaaaataaacgcagggcctttcttgacttgcttttaagtgtgactgatgacgaagggaacaggctaagtcatgaagatattcgagaagaagttgacaccttcatgtttgaggggcacgatacaactgcagctgcaataaactggtccttatacctgttgggttctaacccagaagtccagaaaaaagtggatcatgaattggatgacgtgtttgggaagtctgaccgtcccgctacagtagaagacctgaagaaacttcggtatctggaatgtgttattaaggagacccttcgcctttttccttctgttcctttatttgcccgtagtgttagtgaagattgtgaagtggcaggttacagagttctaaaaggcactgaagccgtcatcattccctatgcattgcacagagatccgagatacttccccaaccccgaggagttccagcctgagcggttcttccccgagaatgcacaagggcgccatccatatgcctacgtgcccttctctgctggccccaggaactgtataggtcaaaagtttgctgtgatggaagaaaagaccattctttcgtgcatcctgaggcacttttggatagaatccaaccagaaaagagaagagcttggtctagaaggacagttgattcttcgtccaagtaatggcatctggatcaagttgaagaggagaaatgcagatgaacgctga

SEQ ID NO: 2 - DNA, 1578 bp, synthetic construct
  source 1..1578: mol_type = other DNA
atggccggactgtggctggggctggtgtggcagaagctgctgctgtggggggccgccagcgctctgtctctggctggagccagcctggtgctgagcctgctgcagagggtggccagctacgctagaaagtggcagcagatgaggcccatccccactgtggcccgggcctaccctctggtgggacacgctctgctgatgaaacccgatggcagagaattcttccagcagattatcgagtataccgaggaatacagacacatgcccctgctgaaactgtgggtgggacctgtgcccatggtggccctgtacaacgccgagaacgtggaagtgatcctgaccagcagcaagcagatcgacaaaagcagcatgtacaaattcctggaaccctggctgggactgggactgctgaccagcactggaaacaagtggagaagcagaagaaagatgctgacccccaccttccacttcaccatcctggaggacttcctggacatcatgaacgaacaggccaacatcctggtgaaaaagctggaaaaacacatcaaccaggaagccttcaactgcttcttctacatcaccctgtgcgccctggacatcatctgcgaaaccgccatgggaaagaacatcggcgcccagagcaacgacgacagcgaatacgtgagagccgtgtatcggatgtccgagatgatcttcagaagaatcaaaatgccctggctgtggctggacctgtggtatctgatgttcaaggagggctgggagcacaagaagagcctgcagatcctgcataccttcaccaacagcgtgatcgccgaaagagccaacgagatgaacgccaacgaggactgccggggagacgggagaggcagcgctcctagcaaaaacaaaagaagagccttcctggacctgctgctgagcgtgaccgacgacgaaggaaatagactgagccacgaagacatcagagaggaagtggacacattcatgttcgaaggccacgacacaacagccgccgccatcaactggtccctgtacctgctgggcagcaaccccgaggtgcagaagaaggtggaccacgagctggacgacgtgtttgggaagagcgacagacctgccaccgtggaggatctgaaaaagctgagatatctggagtgcgtgattaaagagaccctgagactgttcccaagtgtgcccctgttcgccagatctgtgagtgaggactgcgaggtggccggctatagagtgctgaaaggaaccgaggccgtgattattccctacgccctgcacagggaccccaggtacttccccaaccccgaggaattccagcctgagagattctttcccgagaatgctcagggaagacacccctacgcctatgtgcccttcagcgccggccccagaaactgcattggccagaagttcgccgtgatggaggagaagaccatcctgagctgcatcctgagacacttttggattgagagcaaccagaagcgggaggagctgggactggaaggccagctgatcctgagaccaagcaacggaatctggatcaagctgaaaagaagaaacgccgacgagagatga

SEQ ID NO: 3 - DNA, 1578 bp, synthetic construct
  source 1..1578: mol_type = other DNA
atggccggactgtggctgggcctggtgtggcagaagctgctgctgtggggcgccgccagcgctctgtctctggctggagcctccctggtgctgagcctgctgcagagggtggcctcctacgccagaaagtggcagcagatgaggcccatccccacagtggccagggcctaccctctggtgggccacgctctgctgatgaagcctgacggcagggagtttttccagcagatcatcgagtacaccgaggagtacaggcacatgcccctgctgaagctgtgggtgggccccgtgcctatggtggccctgtacaatgccgagaatgtggaggtcattctgacatcctccaagcagatcgataagtcctccatgtacaagtttctggagccttggctgggcctcggcctgctgacaagcacaggcaataagtggaggagcaggagaaagatgctgacccctacctttcactttacaatcctggaggactttctggatatcatgaacgagcaggccaacatcctggtgaagaagctggagaagcacatcaaccaggaggccttcaattgtttcttttacatcacactgtgcgccctggacatcatctgcgagaccgccatgggcaagaatatcggcgcccagtccaacgacgattccgagtacgtgagggccgtgtacaggatgagcgagatgatctttaggagaatcaagatgccctggctgtggctggacctgtggtacctgatgtttaaggagggctgggagcacaagaagagcctgcagatcctgcacacattcacaaattccgtgatcgccgagagagccaacgagatgaatgccaatgaggactgtagaggcgatggcaggggctccgccccctctaagaataagaggagggcctttctggacctgctgctgtccgtgacagatgacgagggcaataggctgtcccacgaggatatcagggaggaggtggacaccttcatgtttgagggccacgacacaacagccgccgccatcaactggagcctgtacctgctgggcagcaatcccgaggtgcagaagaaggtggatcacgagctggatgatgtgttcggcaagagcgacagacccgccacagtggaggatctgaagaagctgagatacctggagtgcgtgatcaaggagacactgagactgttcccttccgtgcccctgttcgccagaagcgtgtccgaggactgtgaggtggccggctacagagtgctgaagggcaccgaggccgtgatcatcccctacgccctgcacagggatcctagatacttccctaaccccgaggagttccagcccgagaggtttttccccgagaatgcccagggcaggcacccctacgcctacgtgccttttagcgccggccctaggaactgtatcggccagaagtttgccgtgatggaggagaagaccatcctgtcctgcatcctgagacacttctggatcgagtccaaccagaagagggaggagctgggcctggagggccagctgatcctgagacctagcaacggcatctggatcaagctgaagagaagaaatgccgatgagagatga

SEQ ID NO: 4 - DNA, 1578 bp, synthetic construct
  source 1..1578: mol_type = other DNA
atggctggactttggctgggactggtgtggcaaaaactgctgctgtggggcgctgctagcgccctgagcctggctggagcttctcttgtgctgagcttactgcaaagagtggccagctatgccagaaagtggcagcagatgagacccatccccaccgtggctagagcttatcctctggtgggacatgctctgctgatgaagcccgacggaagagagtttttccagcagatcatcgagtacaccgaggagtacagacacatgcccctgctgaagctgtgggtgggcccagttcctatggtggctctgtataatgctgaaaacgtggaggtgatcctgaccagcagcaagcagatcgacaagagcagcatgtacaagttcctggagccctggctgggcctgggattactgacatctacaggaaataaatggagaagcagaagaaagatgctgacccccaccttccacttcaccatcctggaggacttcctggacatcatgaacgagcaggccaacatcctggtgaagaagctggagaagcacatcaaccaggaggccttcaactgcttcttctacatcaccctgtgcgccctggacatcatctgcgagacagctatgggcaagaacatcggcgctcaaagcaatgacgacagcgagtatgtgagagccgtgtacagaatgagcgagatgatcttcagaagaatcaagatgccctggctgtggctggacctgtggtatctgatgttcaaggagggctgggagcacaagaagagcctgcagattctgcacaccttcaccaacagcgtgatcgccgagagagccaacgagatgaacgccaacgaggactgcagaggcgacggcagaggatctgctcctagcaaaaataagagaagagccttcctggacctgctgctgagcgtgacagatgacgaaggaaacagactgagccacgaggacatcagagaggaggtggacaccttcatgttcgagggccacgacaccaccgccgctgctattaattggagcctgtatctgctgggaagcaaccccgaagtgcaaaagaaagtggaccacgagctggacgacgtgttcggcaaaagcgacagacccgccacagtggaggatctgaaaaagctgagatacctggagtgcgtgatcaaggagaccctgagactgttccccagcgtgccccttttcgctagatctgtgagcgaagattgcgaggtggccggctatagagtgctgaaaggcaccgaagccgtgatcatcccctacgctctgcacagagaccccagatacttccccaaccccgaggagtttcagcccgagagatttttccccgagaacgcccaaggcagacacccctatgcttatgtgcctttcagcgccggccccagaaattgcattggacaaaagttcgccgtgatggaggagaagaccatcctgagctgcatcctgagacacttctggatcgagagcaaccagaagagagaggagctgggcctggagggccaactgattctgagacctagcaacggcatctggatcaagctgaagagaagaaacgccgacgagagatga

SEQ ID NO: 5 - DNA, 1578 bp, synthetic construct
  source 1..1578: mol_type = other DNA
atggcaggactgtggctgggactggtgtggcagaagctgctgctgtggggagcagcaagcgccctgtccctggcaggcgcctccctggtgctgtctctgctgcagagggtggcaagctacgcaaggaagtggcagcagatgcggcccatccctaccgtggcaagagcatatccactggtgggacacgcactgctgatgaagcctgacggccgcgagttctttcagcagatcatcgagtacacagaggagtatcggcacatgccactgctgaagctgtgggtgggaccagtgccaatggtggccctgtacaacgccgagaatgtggaagtgatcctgaccagctccaagcagatcgataagtctagcatgtataagttcctggagccttggctgggactgggactgctgacctccacaggcaacaagtggaggtctcggagaaagatgctgaccccaacattccactttacaatcctggaggacttcctggatatcatgaacgagcaggccaatatcctggtgaagaagctggagaagcacatcaaccaggaggcctttaattgcttcttttacatcaccctgtgcgccctggacatcatctgtgagacagccatgggcaagaacatcggcgcccagagcaatgacgattccgagtacgtgagggccgtgtatcgcatgtccgagatgatctttaggcgcatcaagatgccctggctgtggctggatctgtggtatctgatgttcaaggagggctgggagcacaagaagagcctgcagatcctgcacacctttacaaactccgtgatcgccgagcgggccaatgagatgaacgccaatgaggactgtaggggcgatggaagaggctctgcccctagcaagaacaagcggagagccttcctggacctgctgctgtctgtgaccgacgatgagggcaatagactgagccacgaggacatcagggaggaggtggatacattcatgtttgagggacacgacaccacagcagcagccatcaactggtctctgtacctgctgggcagcaatccagaggtgcagaagaaggtggatcacgagctggacgacgtgttcggcaagagcgacagacccgccaccgtggaggatctgaagaagctgaggtacctggagtgcgtgatcaaggagacactgagactgttcccctccgtgcctctgtttgccaggtccgtgtctgaggactgtgaggtggccggctatcgcgtgctgaagggcaccgaggccgtgatcatcccttacgccctgcaccgggaccccagatatttccctaacccagaggagtttcagccagagcggttcttcccagagaatgcacagggccggcacccttacgcctatgtgccattctctgccggaccaaggaactgcatcggacagaagtttgccgtgatggaggagaagaccatcctgtcctgtatcctgcgccacttctggatcgagtctaatcagaagagggaggagctgggactggagggacagctgatcctgagaccctccaacggcatctggatcaagctgaagaggcgcaatgccgatgagaggtga

SEQ ID NO: 6 - DNA, 1578 bp, synthetic construct
  source 1..1578: mol_type = other DNA
atggctggactgtggctcggactggtgtggcagaagctgctgctgtggggcgctgcctctgccctcagtctggctggagccagcctggtcctgtctctgctccagagagtggcttcttatgccagaaagtggcagcaaatgaggcctatccctactgtcgccagagcctaccctctggttggacacgccctgctgatgaagcctgacggcagggagttctttcagcagataatcgaatacactgaagaatacagacacatgcctctcctgaaactctgggtgggaccagttcctatggtcgccctgtacaatgccgagaatgtcgaagtgatcctgaccagctcaaagcagattgataagtctagcatgtacaagttcctggagccctggctgggactcggcctgctcacctccaccggcaataagtggcgctcaagaaggaagatgctcactccaacctttcactttaccatactggaggactttctcgacatcatgaatgagcaagccaacattctcgttaagaagctggagaaacacataaaccaggaggctttcaactgtttcttctacataaccctgtgcgctctggacatcatctgcgaaaccgctatgggcaagaacataggagcccaatccaatgacgattctgagtacgtcagggctgtctaccgcatgtctgagatgatcttcagacggattaagatgccctggctgtggctggatctgtggtatctgatgttcaaggaaggatgggagcacaagaagtctctgcaaatcctccatacattcactaacagtgtcattgctgaaagggctaacgagatgaatgctaatgaagattgcagaggagatggacgcggttccgcaccttccaagaacaagcgcagagcatttctcgatctcctgctgagtgttaccgacgacgagggcaaccggctgtcccatgaggacatcagggaagaggtggatacctttatgttcgagggccacgataccaccgcagctgctatcaactggtccctctatctgctgggttccaaccctgaagtgcagaagaaggttgaccacgaactcgatgacgtgtttggtaagagcgacagacctgccaccgtcgaggacctgaagaagctccgctacctggagtgtgtgatcaaggagacactcaggctgtttcctagcgtgcctctgttcgctaggtctgtctctgaggattgcgaggttgccggatacagagtgctcaaaggaactgaggccgtcataatcccttacgccctgcaccgcgaccctagatactttcccaatcccgaggagttccaaccagagagattcttccctgagaatgcccaaggcaggcatccttatgcttatgtcccattctctgccggacctaggaattgtattggacagaagttcgccgttatggaagagaagacaatcctgtcctgtatactgcgccacttctggatcgagagcaatcagaagagggaggaactcggcctggaaggacagctcattctcagaccttcaaacgggatttggatcaaactcaaacggagaaacgccgacgagaggtga

SEQ ID NO: 7 - DNA, 1578 bp, synthetic construct
  source 1..1578: mol_type = other DNA
atggctggtctgtggctgggcctggtgtggcagaagctgctgctgtggggagctgcctctgctctgtccctggctggtgcctccctggtgctgagcctgctgcagagggtggcctcctatgctaggaagtggcagcagatgaggcccatccccacagtggccagggcctaccccctggtgggccatgctctgctgatgaagcctgatggcagggagttcttccagcagatcattgaatacacagaagagtacaggcacatgcctctgctgaagctgtgggtgggccctgttcctatggtggccctgtacaatgctgagaatgttgaggtcattctgacctcctccaagcagattgataagtccagcatgtacaagtttctggagccttggctgggcctgggtctgctgacatccacaggtaataagtggagaagcagaaggaagatgctgacacccacctttcacttcacaatcctggaggacttcctggatatcatgaatgagcaggccaacatcctggtgaagaagctggagaagcacatcaatcaggaggccttcaactgctttttctacatcaccctgtgtgccctggatatcatctgtgaaacagctatgggcaagaatattggtgcccagagcaatgatgactctgaatatgttagggctgtttacagaatgtctgaaatgatctttagaaggatcaagatgccttggctgtggctggatctgtggtacctgatgtttaaggagggctgggagcacaagaagtccctgcagatcctgcacacctttaccaactctgttattgctgagagagccaatgaaatgaatgccaatgaggactgcaggggagatggcagaggctctgctccttccaagaataagagaagagccttcctggatctgctgctgtctgttacagatgatgaaggcaacaggctgagccatgaagacatcagggaggaggtggatacattcatgtttgagggccatgataccacagctgctgctatcaactggtccctgtacctgctgggcagcaaccctgaggtgcagaagaaggtggaccatgaactggatgatgtttttggcaagtctgatagacctgctacagttgaggacctgaagaagctgaggtacctggagtgtgtgatcaaggagacactgaggctgtttccttctgttcccctgtttgccagatctgtttctgaagattgtgaagtggctggttacagggtgctgaagggcacagaggctgttatcatcccctatgctctgcacagagaccccagatactttcctaatcctgaggagtttcagcctgaaaggtttttccctgagaatgctcagggcagacacccctatgcttatgttcccttctctgctggccctaggaactgcattggtcagaagtttgctgtgatggaggagaagaccatcctgagctgcatcctgagacacttctggattgaaagcaaccagaagagggaggagctgggcctggagggccagctgatcctgaggccctccaatggcatctggatcaagctgaagagaagaaatgctgatgagagatga

SEQ ID NO: 8 - DNA, 1578 bp, synthetic construct
  source 1..1578: mol_type = other DNA
atggctggcctgtggctggggctggtctggcagaagctgctcctgtggggggctgcctccgccctctccctggctggggctagcctggtgctgagcctgctgcagagagtggctagctatgctagaaagtggcagcagatgagacccatccccacagtggctagagcctaccccctggtgggccatgccctgctgatgaagcctgatggcagagagttctttcagcagatcattgagtacacagaggagtacagacacatgcccctgctgaagctgtgggtgggccctgtgcctatggtagcactgtacaatgctgagaatgtggaggtgatcctgacaagcagcaagcagattgacaagagcagcatgtacaagttcctggagccctggctgggcctgggcctgctgacaagcaccggcaacaagtggagaagcagaagaaagatgctgacccccaccttccacttcaccatcctggaggacttcctggacatcatgaatgagcaagccaacatcctggtgaagaagctggagaagcacatcaaccaagaggccttcaactgcttcttctacatcaccctgtgtgccctggatatcatctgtgagacagccatgggcaagaacattggggctcagagcaatgatgacagcgagtatgtgagagctgtgtacagaatgagcgagatgatcttcagaagaatcaagatgccctggctgtggctggacctgtggtacctgatgttcaaggagggctgggagcacaagaagagcctgcagatcctgcacaccttcaccaacagcgtgattgctgagagagccaatgagatgaatgccaatgaggactgcagaggggatggcagaggcagcgcccctagcaagaacaagagaagagccttcctggacctgctcctgagcgtgacagatgatgagggcaacagactgagccatgaggacatcagagaagaggtggacaccttcatgtttgagggccatgacaccacagctgctgccatcaactggagcctgtacctgctgggcagcaaccctgaggtgcagaagaaggtggaccatgagctggatgatgtgtttggcaagagcgacagacctgccacagtggaggacctgaagaagctgagatacctggagtgtgtgatcaaggagaccctgagactgttccccagcgtgcccctgtttgctagaagcgtgagcgaggactgtgaggtggctggctacagagtgctgaagggcacagaggctgtgatcatcccctatgccctgcacagagaccctagatacttccccaaccctgaggagtttcagcctgagagattcttccctgagaatgcccaaggcagacacccctatgcctatgtgcccttcagcgctggccctagaaactgcattgggcagaagtttgctgtgatggaggagaagaccatcctgagctgcatcctgagacacttctggattgagagcaatcagaagagagaggagctgggcctggaggggcagctgatcctgagacctagcaatggcatctggatcaagctgaagagaagaaatgctgatgagagatga

SEQ ID NO: 9 - DNA, 1578 bp, synthetic construct
  source 1..1578: mol_type = other DNA
atggctggcctgtggctgggcctggtgtggcagaagctgctgctgtggggagctgccagtgccctgagcctggctggggccagcctggtgctgagcctgctgcagagggtggccagctatgccaggaagtggcagcagatgaggcccatccccacagtggccagggcctaccccctggtgggccatgccctgctgatgaagcctgatggcagggagttcttccagcagatcattgagtacacagaggagtacaggcacatgcccctgctgaagctgtgggtgggccctgtccctatggtggccctgtacaatgctgagaatgtggaggtgatcctgaccagcagcaagcagattgacaagagcagcatgtacaagttcctggagccctggctgggcctgggcctgctgaccagcacaggcaacaagtggaggagcaggaggaagatgctgacccccaccttccacttcaccatcctggaggacttcctggacatcatgaatgagcaggccaacatcctggtgaagaagctggagaagcacatcaaccaggaggccttcaactgcttcttctacatcaccctgtgtgccctggacatcatctgtgagacagccatgggcaagaacattggagcccagagcaatgatgacagtgagtatgtgagggctgtgtacaggatgagtgagatgatcttcaggaggatcaagatgccctggctgtggctggacctgtggtacctgatgttcaaggagggctgggagcacaagaagagcctgcagatcctgcacaccttcaccaacagtgtgattgctgagagggccaatgagatgaatgccaatgaggactgcaggggagatggcaggggcagtgcccccagcaagaacaagaggagagccttcctggacctgctgctgagtgtgacagatgatgagggcaacaggctgagccatgaggacatcagggaggaggtggacaccttcatgtttgagggccatgacaccacagctgcagctatcaactggagcctgtacctgctgggcagcaaccctgaggtgcagaagaaggtggaccatgagctggatgatgtgtttggcaagagtgacaggcctgccacagtggaggacctgaagaagctgaggtacctggagtgtgtgatcaaggagaccctgaggctgttccccagtgtgcccctgtttgccaggagtgtgtctgaggactgtgaggtggctggctacagggtgctgaagggcacagaggctgtgatcatcccctatgccctgcacagggaccccaggtacttccccaaccctgaggagttccagcctgagaggttcttccctgagaatgcccagggcaggcacccctatgcctatgtgcccttcagtgctggccccaggaactgcattggccagaagtttgctgtgatggaggagaagaccatcctgagctgcatcctgaggcacttctggattgagagcaaccagaagagggaggagctgggcctggagggccagctgatcctgaggcccagcaatggcatctggatcaagctgaagaggaggaatgctgatgagaggtga

SEQ ID NO: 10 - DNA, 1578 bp, synthetic construct
  source 1..1578: mol_type = other DNA
atggctggcctgtggctggggctggtctggcagaagctgctcctgtggggggctgcctcagccctctccctggctggggctagcctggtgctgagcctgctgcagagagtggctagctatgctagaaagtggcagcagatgagacccatccccacagtggctagagcctaccccctggtgggccatgccctgctgatgaagcctgatggcagagagttctttcagcagatcattgagtacacagaggagtacagacacatgcccctgctgaagctgtgggtgggccctgtgcctatggtagcactgtacaatgctgagaatgtggaggtgatcctgacaagcagcaagcagattgacaagagcagcatgtacaagttcctggagccctggctgggcctgggcctgctgacaagcactggcaacaagtggagaagcagaagaaagatgctgacccccaccttccacttcaccatcctggaggacttcctggacatcatgaatgagcaagccaacatcctggtgaagaagctggagaagcacatcaaccaagaggccttcaactgcttcttctacatcaccctgtgtgccctggatatcatctgtgagacagccatgggcaagaacattggggctcagagcaatgatgacagtgagtatgtgagagctgtgtacagaatgagtgagatgatcttcagaagaatcaagatgccctggctgtggctggacctgtggtacctgatgttcaaggagggctgggagcacaagaagagcctgcagatcctgcacaccttcaccaactctgtgattgctgagagagccaatgagatgaatgccaatgaggactgcagaggggatggcagaggcagtgcccctagcaagaacaagagaagagccttcctggacctgctcctgtcagtgacagatgatgagggcaacagactgagccatgaggacatcagagaagaggtggacaccttcatgtttgagggccatgacaccacagctgctgccatcaactggagcctgtacctgctgggcagcaaccctgaggtgcagaagaaggtggaccatgagctggatgatgtgtttggcaagagtgacagacctgccacagtggaggacctgaagaagctgagatacctggagtgtgtgatcaaggagaccctgagactgttcccctcagtgcccctgtttgctagaagtgtgagtgaggactgtgaggtggctggctacagagtgctgaagggcacagaggctgtgatcatcccctatgccctgcacagagaccctagatacttccccaaccctgaggagtttcagcctgagagattcttccctgagaatgcccaaggcagacacccctatgcctatgtgcccttcagtgctggccctagaaactgcattgggcagaagtttgctgtgatggaggagaagaccatcctgagctgcatcctgagacacttctggattgagagcaatcagaagagagaggagctgggcctggaggggcagctgatcctgagacctagcaatggcatctggatcaagctgaagagaagaaatgctgatgagagatga

SEQ ID NO: 11 - DNA, 1578 bp, synthetic construct
  source 1..1578: mol_type = other DNA
atggctggactgtggctgggcctggtgtggcagaagctgctgctgtggggagctgccagtgctctgtctctggctggagcctccctggtgctgagcctgctgcagagggtggcctcctatgccagaaagtggcagcagatgaggcccatccccacagtggccagggcctaccctctggtgggccatgctctgctgatgaagcctgatggcagggagtttttccagcagatcattgagtacacagaggagtacaggcacatgcccctgctgaagctgtgggtgggccctgtgcctatggtggccctgtacaatgctgagaatgtggaggtcattctgacatcctccaagcagattgataagtcctccatgtacaagtttctggagccttggctgggcctgggcctgctgacaagcacaggcaataagtggaggagcaggagaaagatgctgacccctacctttcactttacaatcctggaggactttctggatatcatgaatgagcaggccaacatcctggtgaagaagctggagaagcacatcaaccaggaggccttcaattgtttcttctacatcacactgtgtgccctggacatcatctgtgagacagccatgggcaagaatattggagcccagtccaatgatgattctgagtatgtgagggctgtgtacaggatgagtgagatgatctttaggagaatcaagatgccctggctgtggctggacctgtggtacctgatgtttaaggagggctgggagcacaagaagagcctgcagatcctgcacacattcacaaattctgtgattgctgagagagccaatgagatgaatgccaatgaggactgtagaggagatggcaggggctctgccccctctaagaataagaggagggccttcctggacctgctgctgtctgtgacagatgatgagggcaataggctgtcccatgaggatatcagggaggaggtggacaccttcatgtttgagggccatgacacaacagctgctgccatcaactggagcctgtacctgctgggcagcaatcctgaggtgcagaagaaggtggatcatgagctggatgatgtgtttggcaagagtgacagacctgccacagtggaggatctgaagaagctgagatacctggagtgtgtgatcaaggagacactgagactgttcccttctgtgcccctgtttgccagaagtgtgtctgaggactgtgaggtggctggctacagagtgctgaagggcacagaggctgtgatcatcccctatgccctgcacagggatcctagatacttccctaaccctgaggagttccagcctgagaggttcttccctgagaatgcccagggcaggcacccctatgcctatgtgcccttcagtgctggccctaggaactgtattggccagaagtttgctgtgatggaggagaagaccatcctgtcctgcatcctgagacacttctggattgagtccaaccagaagagggaggagctgggcctggagggccagctgatcctgagacctagcaatggcatctggatcaagctgaagagaagaaatgctgatgagagatga

SEQ ID NO: 12 - DNA, 1578 bp, synthetic construct
  source 1..1578: mol_type = other DNA
atggcaggactgtggctgggactggtgtggcagaagctgctgctgtggggagcagcaagtgccctgtccctggcaggagcctccctggtgctgtctctgctgcagagggtggcaagctatgcaaggaagtggcagcagatgaggcccatccctacagtggcaagagcatatccactggtgggacatgcactgctgatgaagcctgatggcagggagttctttcagcagatcattgagtacacagaggagtataggcacatgccactgctgaagctgtgggtgggaccagtgccaatggtggccctgtacaatgctgagaatgtggaagtgatcctgaccagctccaagcagattgataagtctagcatgtataagttcctggagccttggctgggactgggactgctgacctccacaggcaacaagtggaggtctaggagaaagatgctgaccccaacattccactttacaatcctggaggacttcctggatatcatgaatgagcaggccaatatcctggtgaagaagctggagaagcacatcaaccaggaggcctttaattgcttcttctacatcaccctgtgtgccctggacatcatctgtgagacagccatgggcaagaacattggagcccagagcaatgatgattctgagtatgtgagggctgtgtataggatgtctgagatgatctttaggaggatcaagatgccctggctgtggctggatctgtggtatctgatgttcaaggagggctgggagcacaagaagagcctgcagatcctgcacaccttcacaaactctgtgattgctgagagggccaatgagatgaatgccaatgaggactgtaggggagatggaagaggctctgcccctagcaagaacaagaggagagccttcctggacctgctgctgtctgtgacagatgatgagggcaatagactgagccatgaggacatcagggaggaggtggatacattcatgtttgagggacatgacaccacagcagcagccatcaactggtctctgtacctgctgggcagcaatccagaggtgcagaagaaggtggatcatgagctggatgatgtgtttggcaagagtgacagacctgccacagtggaggatctgaagaagctgaggtacctggagtgtgtgatcaaggagacactgagactgttcccctctgtgcctctgtttgccaggtctgtgtctgaggactgtgaggtggctggctatagggtgctgaagggcacagaggctgtgatcatcccttatgccctgcacagggaccccagatatttccctaacccagaggagtttcagccagagaggttcttcccagagaatgcacagggcaggcacccttatgcctatgtgccattctctgctggaccaaggaactgcattggacagaagtttgctgtgatggaggagaagaccatcctgtcctgtatcctgaggcacttctggattgagtctaatcagaagagggaggagctgggactggagggacagctgatcctgagaccctccaatggcatctggatcaagctgaagaggaggaatgctgatgagaggtga

SEQ ID NO: 13 - DNA, 1578 bp, synthetic construct
  source 1..1578: mol_type = other DNA
atggctgggctgtggctggggctggtgtggcagaagctgctgctgtggggagctgctagtgccctcagcctggctggagcaagcctggtgctgagtctcctgcagagagtggcctcctatgccaggaagtggcaacagatgaggcccatcccaacagtggctagagcatacccactggtgggccatgccttgctgatgaagcctgatggcagggagttctttcagcagatcattgaatacactgaggagtacagacacatgcctctgctgaagctgtgggtgggcccagtgcctatggtggccctctacaatgctgagaatgtggaagtgattctgaccagtagcaagcagattgataagagtagcatgtacaagttcctggagccctggctggggttaggcctgctgaccagcacaggaaacaagtggaggagtaggaggaagatgctgacccccaccttccacttcaccatcctggaggactttctggatattatgaatgaacaggcaaacatcttggtgaagaagctggagaagcacattaatcaggaggccttcaactgcttcttctatattaccctgtgtgccctggatatcatctgtgagacagccatgggaaagaatattggagcccagagtaatgatgactctgagtatgtgagggctgtgtacagaatgagtgagatgatcttcaggagaatcaagatgccatggctgtggctggacctgtggtacctgatgttcaaggaggggtgggagcacaagaagtccctgcagattctgcacacctttaccaacagtgtgattgctgagagggccaatgagatgaatgccaatgaggattgcaggggagatggaagaggctctgcccccagcaagaacaaaagaagggccttcctggacctgctgttgtctgtgactgatgatgaagggaacaggctgtcacatgaggacatcagagaggaggtggacaccttcatgtttgagggccatgacaccacagctgctgccattaactggagcctgtacctgctgggaagcaacccagaggtgcagaagaaggtggaccatgagctggatgatgtgtttggcaagtcagacaggcctgccacagtggaggacctgaagaagctgaggtacctggagtgtgtgatcaaggaaacactgaggctgttcccaagtgtgcctctgtttgccaggtctgtgtcagaggactgtgaagtggctggctacagggtgctgaagggaacagaggcagtgatcatcccatatgcactgcacagggaccctaggtattttcctaaccctgaggagttccagccagagaggttctttcctgagaatgctcagggcaggcatccttatgcctatgtgcctttctctgctggccccaggaactgcattggccagaagtttgctgtgatggaagagaagaccattttgtcctgtattctgaggcacttctggattgaatccaaccagaagagggaggagctgggcctggaagggcagctgatcctgaggccctccaatggcatttggatcaaactgaagaggagaaatgctgatgagagatga

SEQ ID NO: 14 - DNA, 1578 bp, synthetic construct
  source 1..1578: mol_type = other DNA
atggctggcctgtggctgggcctggtgtggcagaagctgctcctgtggggagctgcctctgccctgtccctggctggggctagcctggtgctgtctctgttgcagagagtggccagctatgccaggaagtggcaacagatgagacccatccccacagtggccagagcataccccctggtgggccatgccctgctgatgaagcctgatggcagagagttcttccagcagatcattgagtacacagaggaatacagacacatgcctctgctgaagctgtgggtgggccctgtgcctatggtggctctctacaatgctgagaatgtggaagtgatcctgaccagcagcaagcagattgacaagtctagcatgtacaagttcctggagccttggctgggactgggactgctgaccagcacaggcaacaagtggaggagcagaagaaagatgctgacacctaccttccactttaccatcctggaagatttcctggacattatgaatgagcaggccaacatcctggtcaagaagctggaaaagcacatcaaccaggaggcttttaactgcttcttctacatcaccctgtgtgccctggacatcatctgtgagacagccatgggcaagaacattggagctcagagcaatgatgatagtgaatatgttagagctgtctacaggatgagtgagatgatcttcaggagaatcaagatgccctggctgtggctggatctgtggtacctgatgttcaaggaaggatgggagcacaagaaaagcctgcagatcctgcacaccttcaccaacagtgtgattgctgagagagccaatgagatgaatgccaatgaggactgtagaggagatggcaggggcagtgccccttctaagaacaagagaagggccttcctggacctgctgctgagtgttacagatgatgagggcaatagactgtctcatgaggatatcagagaagaggtggacaccttcatgtttgagggccatgacactacagctgctgctattaactggtccctgtacctgctgggcagcaaccctgaagtgcagaagaaggtggaccatgagctggatgatgtgtttggaaagagtgatagacctgccacagtggaagacctgaagaagctaaggtacctggagtgtgtgatcaaggaaacactgagactgttcccctctgtgcctctgtttgccagaagtgtgtctgaggattgtgaggtggctggctacagggtgctgaagggcacagaggctgtgatcatcccttatgccctgcacagggaccccagatatttccctaaccctgaggagttccagcctgagaggttcttcccagagaatgcccagggcagacatccttatgcctatgtgccattcagtgctggtcctagaaactgcattggccagaagtttgctgtgatggaagagaagaccatcctgagctgcatcctgaggcacttctggattgagtctaatcagaagagagaggaactgggcctggaaggccagctgatcctcaggccaagcaatggcatctggatcaagctgaagaggaggaatgctgatgagaggtga

SEQ ID NO: 15 - DNA, 1578 bp, synthetic construct
  source 1..1578: mol_type = other DNA
atggctggactgtggctgggactggtgtggcagaaactgctgctttggggagctgcctctgctctgtctcttgctggtgcttctctggtgctgagcctgctgcagagagtggcctcttatgccagaaagtggcagcagatgaggcccattcctacagtggccagagcctatcctcttgtgggacatgccctgctgatgaagcctgatggcagagagttcttccagcagatcattgagtacacagaggaatacaggcacatgcccctgctcaagctgtgggttggacctgtgcctatggtggccctgtacaatgctgagaatgtggaagtgatcctgaccagcagcaagcagattgacaagtccagcatgtacaagtttctggaaccctggctgggcctgggcctgctgacatctactggaaacaagtggaggagcaggaggaagatgctgacccctaccttccactttacaattctggaggacttcctggacatcatgaatgagcaggccaacatcctggtcaagaagctggaaaagcacatcaatcaagaggccttcaactgcttcttctacatcaccctgtgtgccctggatatcatctgtgagacagccatgggcaagaacattggagcccagagcaatgatgatagtgaatatgtgagggctgtgtacaggatgagtgagatgatcttcaggaggatcaagatgccttggctgtggctggacctgtggtatctgatgttcaaagaaggctgggagcacaagaagtccctgcagatcctgcacaccttcaccaacagtgtgattgctgagagggccaatgagatgaatgccaatgaagattgcagaggagatggcaggggaagtgcccctagcaagaacaagagaagggccttcctggatctgctgctgagtgtgacagatgatgagggcaatagactgagccatgaggacatcagagaagaggtggacaccttcatgtttgagggccatgatacaacagctgctgccatcaattggagcctgtacctgctgggcagcaaccctgaggtgcagaagaaggtggaccatgagctggatgatgtgtttggcaagtctgatagacctgccacagtggaagatctgaagaagctgagatacctggaatgtgtgatcaaagagacactgaggctgttccctagtgtgcccctgtttgccagaagtgtgtctgaggattgtgaagtggctggctacagagtgctgaagggaacagaggctgtgatcatcccctatgctctgcacagagatcccaggtacttccccaatcctgaagagttccagcctgagaggttcttcccagagaatgcccagggcagacacccctatgcctatgtgccattctctgctggacctaggaactgcattggccagaagtttgctgtgatggaagagaaaaccatcctgagctgtatcctgaggcacttctggattgagagcaaccagaagagagaggaactgggcctggagggacagctgattctgaggcctagcaatggcatctggatcaagctgaagaggagaaatgctgatgagaggtga

SEQ ID NO: 16 - DNA, 1578 bp, synthetic construct
  source 1..1578: mol_type = other DNA
atggctggactttggctgggactggtgtggcagaagctgctgctgtggggagctgctagtgccctgagcctggctggagcttctcttgtgctgagcttactgcaaagagtggccagctatgccagaaagtggcagcagatgagacccatccccacagtggctagagcttatcctctggtgggacatgctctgctgatgaagcctgatggaagagagtttttccagcagatcattgagtacacagaggagtacagacacatgcccctgctgaagctgtgggtgggcccagttcctatggtggctctgtataatgctgagaatgtggaggtgatcctgaccagcagcaagcagattgacaagagcagcatgtacaagttcctggagccctggctgggcctgggattactgacatctacaggaaataaatggagaagcagaagaaagatgctgacccccaccttccacttcaccatcctggaggacttcctggacatcatgaatgagcaggccaacatcctggtgaagaagctggagaagcacatcaaccaggaggccttcaactgcttcttctacatcaccctgtgtgccctggacatcatctgtgagacagctatgggcaagaacattggagctcagagcaatgatgacagtgagtatgtgagagctgtgtacagaatgagtgagatgatcttcagaagaatcaagatgccctggctgtggctggacctgtggtatctgatgttcaaggagggctgggagcacaagaagagcctgcagatcctgcacaccttcaccaacagtgtgattgctgagagagccaatgagatgaatgccaatgaggactgcagaggagatggcagaggatctgctcctagcaaaaataagagaagagccttcctggacctgctgctgagtgtgacagatgatgaaggaaacagactgagccatgaggacatcagagaggaggtggacaccttcatgtttgagggccatgacaccacagctgctgctattaattggagcctgtatctgctgggaagcaaccctgaagtgcagaagaaggtggaccatgagctggatgatgtgtttggcaagagtgacagacctgccacagtggaggatctgaagaagctgagatacctggagtgtgtgatcaaggagaccctgagactgttccccagtgtgcccctgtttgctagatctgtgagtgaagattgtgaggtggctggctatagagtgctgaaaggcacagaagctgtgatcatcccctatgctctgcacagagaccccagatacttccccaaccctgaggagtttcagcctgagagattcttccctgagaatgcccaaggcagacacccctatgcttatgtgcccttcagtgctggccccagaaattgcattggacagaagtttgctgtgatggaggagaagaccatcctgagctgcatcctgagacacttctggattgagagcaaccagaagagagaggagctgggcctggagggccaactgattctgagacctagcaatggcatctggatcaagctgaagagaaggaatgctgatgagagatga

SEQ ID NO: 17 - DNA, 1578 bp, synthetic construct
  source 1..1578: mol_type = other DNA
atggctggactgtggctgggactggtgtggcagaaactgctgctgtggggagctgcttctgcactgtctctggctggggcatctctggtcctgtccctgctgcagagagtggcctcctatgccagaaagtggcagcagatgagacctatccccacagtggccagggcataccccctggtgggtcatgccctcctgatgaaaccagatgggagggaatttttccagcagattattgagtacacagaagaatatagacacatgcccctcctcaagctgtgggttgggcctgtccctatggttgccctctataatgctgagaatgttgaagttattctcacatcatctaagcagattgataagagtagcatgtataagtttctggaaccctggctggggctggggctcctcacctcaactgggaacaagtggaggtctagaagaaaaatgctcacccccaccttccatttcaccatcctggaggactttctggacatcatgaatgagcaggctaacatcctggttaaaaaactggagaaacatatcaatcaggaagccttcaattgtttcttctatattactctctgtgctctggacattatctgtgagactgcaatgggaaagaatattggtgcacagtcaaatgatgattcagaatatgtcagggctgtctacagaatgtctgagatgatctttaggagaatcaagatgccctggctgtggctggatctgtggtacctcatgttcaaggaaggatgggagcataagaaaagtctgcagattctgcataccttcaccaactcagtgattgcagagagagcaaatgagatgaatgctaatgaagattgcaggggggatggaaggggaagtgcccctagcaaaaataagagaagggcctttctggatctcctcctgagtgttacagatgatgagggtaatagactcagccatgaagatatcagagaagaggtggacacattcatgtttgagggccatgatactactgcagcagctattaactggagtctgtacctgctgggctcaaaccctgaggttcagaagaaagttgaccatgaactggatgatgtctttgggaagtcagacaggcctgctactgtggaagatctgaagaagctgaggtacctggagtgcgtcattaaggaaaccctgagactcttcccctctgtccccctgtttgctagatcagtttcagaagattgtgaggtggctggctatagggtcctgaagggtactgaggcagtgattatcccttatgctctccacagggatcctaggtactttccaaatcctgaggagttccagcctgagagatttttcccagaaaatgcccagggcaggcatccctatgcttatgtcccctttagtgcaggaccaagaaactgtattggccagaaatttgcagtcatggaggagaaaacaatcctgtcttgcatcctgaggcatttctggattgaatccaaccagaaaagagaggagctgggactggaagggcagctgattctgagaccaagcaatgggatttggatcaagctgaagaggaggaatgcagatgagaggtaa

SEQ ID NO: 18 - DNA, 4325 bp, synthetic construct
  source 1..4325: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgagacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccatctccagcctcggggctgtccgcagggggacggctgccttcgggggggacggggcagggcggggttcggcttctggcgtgtgaccggcggctctagtgcctctgctaaccatgttcatgccttcttctttttcctacagctcctgggcaacgtgctggttattgtgctgtctcatcattttggcaaaaagcttgccaccatggcggggctctggctggggctcgtgtggcagaagctgctgctgtggggcgcggcgagtgccctttccctggccggcgccagtctggtcctgagcctgctgcagagggtggcgagctacgcgcggaaatggcagcagatgcggcccatccccacggtggcccgcgcctacccactggtgggccacgcgctgctgatgaagccggacgggcgagaattttttcagcagatcattgagtacacagaggaataccgccacatgccgctgctgaagctctgggtcgggccagtgcccatggtggccctttataatgcagaaaatgtggaggtaattttaactagttcaaagcaaattgacaaatcctctatgtacaagtttttagaaccatggcttggcctaggacttcttacaagtactggaaacaaatggcgctccaggagaaagatgttaacacccactttccattttaccattctggaagatttcttagatatcatgaatgaacaagcaaatatattggttaagaaacttgaaaaacacattaaccaagaagcatttaactgctttttttacatcactctttgtgccttagatatcatctgtgaaacagctatggggaagaatattggtgctcaaagtaatgatgattccgagtatgtccgtgcagtttatagaatgagtgagatgatatttcgaagaataaagatgccctggctttggcttgatctctggtaccttatgtttaaagaaggatgggaacacaaaaagagccttcagatcctacatacttttaccaacagtgtcatcgctgaacgggccaatgaaatgaacgccaatgaagactgtagaggtgatggcaggggctctgccccctccaaaaataaacgcagggcctttcttgacttgcttttaagtgtgactgatgacgaagggaacaggctaagtcatgaagatattcgagaagaagttgacaccttcatgtttgaggggcacgatacaactgcagctgcaataaactggtccttatacctgttgggttctaacccagaagtccagaaaaaagtggatcatgaattggatgacgtgtttgggaagtctgaccgtcccgctacagtagaagacctgaagaaacttcggtatctggaatgtgttattaaggagacccttcgcctttttccttctgttcctttatttgcccgtagtgttagtgaagattgtgaagtggcaggttacagagttctaaaaggcactgaagccgtcatcattccctatgcattgcacagagatccgagatacttccccaaccccgaggagttccagcctgagcggttcttccccgagaatgcacaagggcgccatccatatgcctacgtgcccttctctgctggccccaggaactgtataggtcaaaagtttgctgtgatggaagaaaagaccattctttcgtgcatcctgaggcacttttggatagaatccaaccagaaaagagaagagcttggtctagaaggacagttgattcttcgtccaagtaatggcatctggatcaagttgaagaggagaaatgcagatgaacgctgagaattcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtttattgcagcttataatggttacaaataaagcaatagcatcacaaatttcacaaataaagcatttttttcactgcattctagttgtggtttgtccaaactcatcaatgtatcttagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 19 - DNA, 4325 bp, synthetic construct
  source 1..4325: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgagacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccatctccagcctcggggctgtccgcagggggacggctgccttcgggggggacggggcagggcggggttcggcttctggcgtgtgaccggcggctctagtgcctctgctaaccatgttcatgccttcttctttttcctacagctcctgggcaacgtgctggttattgtgctgtctcatcattttggcaaaaagcttgccaccatggccggactgtggctggggctggtgtggcagaagctgctgctgtggggggccgccagcgctctgtctctggctggagccagcctggtgctgagcctgctgcagagggtggccagctacgctagaaagtggcagcagatgaggcccatccccactgtggcccgggcctaccctctggtgggacacgctctgctgatgaaacccgatggcagagaattcttccagcagattatcgagtataccgaggaatacagacacatgcccctgctgaaactgtgggtgggacctgtgcccatggtggccctgtacaacgccgagaacgtggaagtgatcctgaccagcagcaagcagatcgacaaaagcagcatgtacaaattcctggaaccctggctgggactgggactgctgaccagcactggaaacaagtggagaagcagaagaaagatgctgacccccaccttccacttcaccatcctggaggacttcctggacatcatgaacgaacaggccaacatcctggtgaaaaagctggaaaaacacatcaaccaggaagccttcaactgcttcttctacatcaccctgtgcgccctggacatcatctgcgaaaccgccatgggaaagaacatcggcgcccagagcaacgacgacagcgaatacgtgagagccgtgtatcggatgtccgagatgatcttcagaagaatcaaaatgccctggctgtggctggacctgtggtatctgatgttcaaggagggctgggagcacaagaagagcctgcagatcctgcataccttcaccaacagcgtgatcgccgaaagagccaacgagatgaacgccaacgaggactgccggggagacgggagaggcagcgctcctagcaaaaacaaaagaagagccttcctggacctgctgctgagcgtgaccgacgacgaaggaaatagactgagccacgaagacatcagagaggaagtggacacattcatgttcgaaggccacgacacaacagccgccgccatcaactggtccctgtacctgctgggcagcaaccccgaggtgcagaagaaggtggaccacgagctggacgacgtgtttgggaagagcgacagacctgccaccgtggaggatctgaaaaagctgagatatctggagtgcgtgattaaagagaccctgagactgttcccaagtgtgcccctgttcgccagatctgtgagtgaggactgcgaggtggccggctatagagtgctgaaaggaaccgaggccgtgattattccctacgccctgcacagggaccccaggtacttccccaaccccgaggaattccagcctgagagattctttcccgagaatgctcagggaagacacccctacgcctatgtgcccttcagcgccggccccagaaactgcattggccagaagttcgccgtgatggaggagaagaccatcctgagctgcatcctgagacacttttggattgagagcaaccagaagcgggaggagctgggactggaaggccagctgatcctgagaccaagcaacggaatctggatcaagctgaaaagaagaaacgccgacgagagatgagaattcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtttattgcagcttataatggttacaaataaagcaatagcatcacaaatttcacaaataaagcatttttttcactgcattctagttgtggtttgtccaaactcatcaatgtatcttagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 20 - DNA, 4325 bp, synthetic construct
  source 1..4325: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgagacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccatctccagcctcggggctgtccgcagggggacggctgccttcgggggggacggggcagggcggggttcggcttctggcgtgtgaccggcggctctagtgcctctgctaaccatgttcatgccttcttctttttcctacagctcctgggcaacgtgctggttattgtgctgtctcatcattttggcaaaaagcttgccaccatggccggactgtggctgggcctggtgtggcagaagctgctgctgtggggcgccgccagcgctctgtctctggctggagcctccctggtgctgagcctgctgcagagggtggcctcctacgccagaaagtggcagcagatgaggcccatccccacagtggccagggcctaccctctggtgggccacgctctgctgatgaagcctgacggcagggagtttttccagcagatcatcgagtacaccgaggagtacaggcacatgcccctgctgaagctgtgggtgggccccgtgcctatggtggccctgtacaatgccgagaatgtggaggtcattctgacatcctccaagcagatcgataagtcctccatgtacaagtttctggagccttggctgggcctcggcctgctgacaagcacaggcaataagtggaggagcaggagaaagatgctgacccctacctttcactttacaatcctggaggactttctggatatcatgaacgagcaggccaacatcctggtgaagaagctggagaagcacatcaaccaggaggccttcaattgtttcttttacatcacactgtgcgccctggacatcatctgcgagaccgccatgggcaagaatatcggcgcccagtccaacgacgattccgagtacgtgagggccgtgtacaggatgagcgagatgatctttaggagaatcaagatgccctggctgtggctggacctgtggtacctgatgtttaaggagggctgggagcacaagaagagcctgcagatcctgcacacattcacaaattccgtgatcgccgagagagccaacgagatgaatgccaatgaggactgtagaggcgatggcaggggctccgccccctctaagaataagaggagggcctttctggacctgctgctgtccgtgacagatgacgagggcaataggctgtcccacgaggatatcagggaggaggtggacaccttcatgtttgagggccacgacacaacagccgccgccatcaactggagcctgtacctgctgggcagcaatcccgaggtgcagaagaaggtggatcacgagctggatgatgtgttcggcaagagcgacagacccgccacagtggaggatctgaagaagctgagatacctggagtgcgtgatcaaggagacactgagactgttcccttccgtgcccctgttcgccagaagcgtgtccgaggactgtgaggtggccggctacagagtgctgaagggcaccgaggccgtgatcatcccctacgccctgcacagggatcctagatacttccctaaccccgaggagttccagcccgagaggtttttccccgagaatgcccagggcaggcacccctacgcctacgtgccttttagcgccggccctaggaactgtatcggccagaagtttgccgtgatggaggagaagaccatcctgtcctgcatcctgagacacttctggatcgagtccaaccagaagagggaggagctgggcctggagggccagctgatcctgagacctagcaacggcatctggatcaagctgaagagaagaaatgccgatgagagatgagaattcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtttattgcagcttataatggttacaaataaagcaatagcatcacaaatttcacaaataaagcatttttttcactgcattctagttgtggtttgtccaaactcatcaatgtatcttagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 21 - DNA, 4325 bp, synthetic construct
  source 1..4325: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgagacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccatctccagcctcggggctgtccgcagggggacggctgccttcgggggggacggggcagggcggggttcggcttctggcgtgtgaccggcggctctagtgcctctgctaaccatgttcatgccttcttctttttcctacagctcctgggcaacgtgctggttattgtgctgtctcatcattttggcaaaaagcttgccaccatggctggactttggctgggactggtgtggcaaaaactgctgctgtggggcgctgctagcgccctgagcctggctggagcttctcttgtgctgagcttactgcaaagagtggccagctatgccagaaagtggcagcagatgagacccatccccaccgtggctagagcttatcctctggtgggacatgctctgctgatgaagcccgacggaagagagtttttccagcagatcatcgagtacaccgaggagtacagacacatgcccctgctgaagctgtgggtgggcccagttcctatggtggctctgtataatgctgaaaacgtggaggtgatcctgaccagcagcaagcagatcgacaagagcagcatgtacaagttcctggagccctggctgggcctgggattactgacatctacaggaaataaatggagaagcagaagaaagatgctgacccccaccttccacttcaccatcctggaggacttcctggacatcatgaacgagcaggccaacatcctggtgaagaagctggagaagcacatcaaccaggaggccttcaactgcttcttctacatcaccctgtgcgccctggacatcatctgcgagacagctatgggcaagaacatcggcgctcaaagcaatgacgacagcgagtatgtgagagccgtgtacagaatgagcgagatgatcttcagaagaatcaagatgccctggctgtggctggacctgtggtatctgatgttcaaggagggctgggagcacaagaagagcctgcagattctgcacaccttcaccaacagcgtgatcgccgagagagccaacgagatgaacgccaacgaggactgcagaggcgacggcagaggatctgctcctagcaaaaataagagaagagccttcctggacctgctgctgagcgtgacagatgacgaaggaaacagactgagccacgaggacatcagagaggaggtggacaccttcatgttcgagggccacgacaccaccgccgctgctattaattggagcctgtatctgctgggaagcaaccccgaagtgcaaaagaaagtggaccacgagctggacgacgtgttcggcaaaagcgacagacccgccacagtggaggatctgaaaaagctgagatacctggagtgcgtgatcaaggagaccctgagactgttccccagcgtgccccttttcgctagatctgtgagcgaagattgcgaggtggccggctatagagtgctgaaaggcaccgaagccgtgatcatcccctacgctctgcacagagaccccagatacttccccaaccccgaggagtttcagcccgagagatttttccccgagaacgcccaaggcagacacccctatgcttatgtgcctttcagcgccggccccagaaattgcattggacaaaagttcgccgtgatggaggagaagaccatcctgagctgcatcctgagacacttctggatcgagagcaaccagaagagagaggagctgggcctggagggccaactgattctgagacctagcaacggcatctggatcaagctgaagagaagaaacgccgacgagagatgagaattcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtttattgcagcttataatggttacaaataaagcaatagcatcacaaatttcacaaataaagcatttttttcactgcattctagttgtggtttgtccaaactcatcaatgtatcttagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 22 - DNA, 4325 bp, synthetic construct
  source 1..4325: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgagacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccatctccagcctcggggctgtccgcagggggacggctgccttcgggggggacggggcagggcggggttcggcttctggcgtgtgaccggcggctctagtgcctctgctaaccatgttcatgccttcttctttttcctacagctcctgggcaacgtgctggttattgtgctgtctcatcattttggcaaaaagcttgccaccatggcaggactgtggctgggactggtgtggcagaagctgctgctgtggggagcagcaagcgccctgtccctggcaggcgcctccctggtgctgtctctgctgcagagggtggcaagctacgcaaggaagtggcagcagatgcggcccatccctaccgtggcaagagcatatccactggtgggacacgcactgctgatgaagcctgacggccgcgagttctttcagcagatcatcgagtacacagaggagtatcggcacatgccactgctgaagctgtgggtgggaccagtgccaatggtggccctgtacaacgccgagaatgtggaagtgatcctgaccagctccaagcagatcgataagtctagcatgtataagttcctggagccttggctgggactgggactgctgacctccacaggcaacaagtggaggtctcggagaaagatgctgaccccaacattccactttacaatcctggaggacttcctggatatcatgaacgagcaggccaatatcctggtgaagaagctggagaagcacatcaaccaggaggcctttaattgcttcttttacatcaccctgtgcgccctggacatcatctgtgagacagccatgggcaagaacatcggcgcccagagcaatgacgattccgagtacgtgagggccgtgtatcgcatgtccgagatgatctttaggcgcatcaagatgccctggctgtggctggatctgtggtatctgatgttcaaggagggctgggagcacaagaagagcctgcagatcctgcacacctttacaaactccgtgatcgccgagcgggccaatgagatgaacgccaatgaggactgtaggggcgatggaagaggctctgcccctagcaagaacaagcggagagccttcctggacctgctgctgtctgtgaccgacgatgagggcaatagactgagccacgaggacatcagggaggaggtggatacattcatgtttgagggacacgacaccacagcagcagccatcaactggtctctgtacctgctgggcagcaatccagaggtgcagaagaaggtggatcacgagctggacgacgtgttcggcaagagcgacagacccgccaccgtggaggatctgaagaagctgaggtacctggagtgcgtgatcaaggagacactgagactgttcccctccgtgcctctgtttgccaggtccgtgtctgaggactgtgaggtggccggctatcgcgtgctgaagggcaccgaggccgtgatcatcccttacgccctgcaccgggaccccagatatttccctaacccagaggagtttcagccagagcggttcttcccagagaatgcacagggccggcacccttacgcctatgtgccattctctgccggaccaaggaactgcatcggacagaagtttgccgtgatggaggagaagaccatcctgtcctgtatcctgcgccacttctggatcgagtctaatcagaagagggaggagctgggactggagggacagctgatcctgagaccctccaacggcatctggatcaagctgaagaggcgcaatgccgatgagaggtgagaattcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtttattgcagcttataatggttacaaataaagcaatagcatcacaaatttcacaaataaagcatttttttcactgcattctagttgtggtttgtccaaactcatcaatgtatcttagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 23 - DNA, 4325 bp, synthetic construct
  source 1..4325: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgagacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccatctccagcctcggggctgtccgcagggggacggctgccttcgggggggacggggcagggcggggttcggcttctggcgtgtgaccggcggctctagtgcctctgctaaccatgttcatgccttcttctttttcctacagctcctgggcaacgtgctggttattgtgctgtctcatcattttggcaaaaagcttgccaccatggctggactgtggctcggactggtgtggcagaagctgctgctgtggggcgctgcctctgccctcagtctggctggagccagcctggtcctgtctctgctccagagagtggcttcttatgccagaaagtggcagcaaatgaggcctatccctactgtcgccagagcctaccctctggttggacacgccctgctgatgaagcctgacggcagggagttctttcagcagataatcgaatacactgaagaatacagacacatgcctctcctgaaactctgggtgggaccagttcctatggtcgccctgtacaatgccgagaatgtcgaagtgatcctgaccagctcaaagcagattgataagtctagcatgtacaagttcctggagccctggctgggactcggcctgctcacctccaccggcaataagtggcgctcaagaaggaagatgctcactccaacctttcactttaccatactggaggactttctcgacatcatgaatgagcaagccaacattctcgttaagaagctggagaaacacataaaccaggaggctttcaactgtttcttctacataaccctgtgcgctctggacatcatctgcgaaaccgctatgggcaagaacataggagcccaatccaatgacgattctgagtacgtcagggctgtctaccgcatgtctgagatgatcttcagacggattaagatgccctggctgtggctggatctgtggtatctgatgttcaaggaaggatgggagcacaagaagtctctgcaaatcctccatacattcactaacagtgtcattgctgaaagggctaacgagatgaatgctaatgaagattgcagaggagatggacgcggttccgcaccttccaagaacaagcgcagagcatttctcgatctcctgctgagtgttaccgacgacgagggcaaccggctgtcccatgaggacatcagggaagaggtggatacctttatgttcgagggccacgataccaccgcagctgctatcaactggtccctctatctgctgggttccaaccctgaagtgcagaagaaggttgaccacgaactcgatgacgtgtttggtaagagcgacagacctgccaccgtcgaggacctgaagaagctccgctacctggagtgtgtgatcaaggagacactcaggctgtttcctagcgtgcctctgttcgctaggtctgtctctgaggattgcgaggttgccggatacagagtgctcaaaggaactgaggccgtcataatcccttacgccctgcaccgcgaccctagatactttcccaatcccgaggagttccaaccagagagattcttccctgagaatgcccaaggcaggcatccttatgcttatgtcccattctctgccggacctaggaattgtattggacagaagttcgccgttatggaagagaagacaatcctgtcctgtatactgcgccacttctggatcgagagcaatcagaagagggaggaactcggcctggaaggacagctcattctcagaccttcaaacgggatttggatcaaactcaaacggagaaacgccgacgagaggtgagaattcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtttattgcagcttataatggttacaaataaagcaatagcatcacaaatttcacaaataaagcatttttttcactgcattctagttgtggtttgtccaaactcatcaatgtatcttagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 24 - DNA, 4325 bp, synthetic construct
  source 1..4325: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgagacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccatctccagcctcggggctgtccgcagggggacggctgccttcgggggggacggggcagggcggggttcggcttctggcgtgtgaccggcggctctagtgcctctgctaaccatgttcatgccttcttctttttcctacagctcctgggcaacgtgctggttattgtgctgtctcatcattttggcaaaaagcttgccaccatggctggtctgtggctgggcctggtgtggcagaagctgctgctgtggggagctgcctctgctctgtccctggctggtgcctccctggtgctgagcctgctgcagagggtggcctcctatgctaggaagtggcagcagatgaggcccatccccacagtggccagggcctaccccctggtgggccatgctctgctgatgaagcctgatggcagggagttcttccagcagatcattgaatacacagaagagtacaggcacatgcctctgctgaagctgtgggtgggccctgttcctatggtggccctgtacaatgctgagaatgttgaggtcattctgacctcctccaagcagattgataagtccagcatgtacaagtttctggagccttggctgggcctgggtctgctgacatccacaggtaataagtggagaagcagaaggaagatgctgacacccacctttcacttcacaatcctggaggacttcctggatatcatgaatgagcaggccaacatcctggtgaagaagctggagaagcacatcaatcaggaggccttcaactgctttttctacatcaccctgtgtgccctggatatcatctgtgaaacagctatgggcaagaatattggtgcccagagcaatgatgactctgaatatgttagggctgtttacagaatgtctgaaatgatctttagaaggatcaagatgccttggctgtggctggatctgtggtacctgatgtttaaggagggctgggagcacaagaagtccctgcagatcctgcacacctttaccaactctgttattgctgagagagccaatgaaatgaatgccaatgaggactgcaggggagatggcagaggctctgctccttccaagaataagagaagagccttcctggatctgctgctgtctgttacagatgatgaaggcaacaggctgagccatgaagacatcagggaggaggtggatacattcatgtttgagggccatgataccacagctgctgctatcaactggtccctgtacctgctgggcagcaaccctgaggtgcagaagaaggtggaccatgaactggatgatgtttttggcaagtctgatagacctgctacagttgaggacctgaagaagctgaggtacctggagtgtgtgatcaaggagacactgaggctgtttccttctgttcccctgtttgccagatctgtttctgaagattgtgaagtggctggttacagggtgctgaagggcacagaggctgttatcatcccctatgctctgcacagagaccccagatactttcctaatcctgaggagtttcagcctgaaaggtttttccctgagaatgctcagggcagacacccctatgcttatgttcccttctctgctggccctaggaactgcattggtcagaagtttgctgtgatggaggagaagaccatcctgagctgcatcctgagacacttctggattgaaagcaaccagaagagggaggagctgggcctggagggccagctgatcctgaggccctccaatggcatctggatcaagctgaagagaagaaatgctgatgagagatgagaattcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtttattgcagcttataatggttacaaataaagcaatagcatcacaaatttcacaaataaagcatttttttcactgcattctagttgtggtttgtccaaactcatcaatgtatcttagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 25 - DNA, 4325 bp, synthetic construct
  source 1..4325: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgagacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccatctccagcctcggggctgtccgcagggggacggctgccttcgggggggacggggcagggcggggttcggcttctggcgtgtgaccggcggctctagtgcctctgctaaccatgttcatgccttcttctttttcctacagctcctgggcaacgtgctggttattgtgctgtctcatcattttggcaaaaagcttgccaccatggctggcctgtggctggggctggtctggcagaagctgctcctgtggggggctgcctccgccctctccctggctggggctagcctggtgctgagcctgctgcagagagtggctagctatgctagaaagtggcagcagatgagacccatccccacagtggctagagcctaccccctggtgggccatgccctgctgatgaagcctgatggcagagagttctttcagcagatcattgagtacacagaggagtacagacacatgcccctgctgaagctgtgggtgggccctgtgcctatggtagcactgtacaatgctgagaatgtggaggtgatcctgacaagcagcaagcagattgacaagagcagcatgtacaagttcctggagccctggctgggcctgggcctgctgacaagcaccggcaacaagtggagaagcagaagaaagatgctgacccccaccttccacttcaccatcctggaggacttcctggacatcatgaatgagcaagccaacatcctggtgaagaagctggagaagcacatcaaccaagaggccttcaactgcttcttctacatcaccctgtgtgccctggatatcatctgtgagacagccatgggcaagaacattggggctcagagcaatgatgacagcgagtatgtgagagctgtgtacagaatgagcgagatgatcttcagaagaatcaagatgccctggctgtggctggacctgtggtacctgatgttcaaggagggctgggagcacaagaagagcctgcagatcctgcacaccttcaccaacagcgtgattgctgagagagccaatgagatgaatgccaatgaggactgcagaggggatggcagaggcagcgcccctagcaagaacaagagaagagccttcctggacctgctcctgagcgtgacagatgatgagggcaacagactgagccatgaggacatcagagaagaggtggacaccttcatgtttgagggccatgacaccacagctgctgccatcaactggagcctgtacctgctgggcagcaaccctgaggtgcagaagaaggtggaccatgagctggatgatgtgtttggcaagagcgacagacctgccacagtggaggacctgaagaagctgagatacctggagtgtgtgatcaaggagaccctgagactgttccccagcgtgcccctgtttgctagaagcgtgagcgaggactgtgaggtggctggctacagagtgctgaagggcacagaggctgtgatcatcccctatgccctgcacagagaccctagatacttccccaaccctgaggagtttcagcctgagagattcttccctgagaatgcccaaggcagacacccctatgcctatgtgcccttcagcgctggccctagaaactgcattgggcagaagtttgctgtgatggaggagaagaccatcctgagctgcatcctgagacacttctggattgagagcaatcagaagagagaggagctgggcctggaggggcagctgatcctgagacctagcaatggcatctggatcaagctgaagagaagaaatgctgatgagagatgagaattcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtttattgcagcttataatggttacaaataaagcaatagcatcacaaatttcacaaataaagcatttttttcactgcattctagttgtggtttgtccaaactcatcaatgtatcttagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 26 - DNA, 4325 bp, synthetic construct
  source 1..4325: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgagacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccatctccagcctcggggctgtccgcagggggacggctgccttcgggggggacggggcagggcggggttcggcttctggcgtgtgaccggcggctctagtgcctctgctaaccatgttcatgccttcttctttttcctacagctcctgggcaacgtgctggttattgtgctgtctcatcattttggcaaaaagcttgccaccatggctggcctgtggctgggcctggtgtggcagaagctgctgctgtggggagctgccagtgccctgagcctggctggggccagcctggtgctgagcctgctgcagagggtggccagctatgccaggaagtggcagcagatgaggcccatccccacagtggccagggcctaccccctggtgggccatgccctgctgatgaagcctgatggcagggagttcttccagcagatcattgagtacacagaggagtacaggcacatgcccctgctgaagctgtgggtgggccctgtccctatggtggccctgtacaatgctgagaatgtggaggtgatcctgaccagcagcaagcagattgacaagagcagcatgtacaagttcctggagccctggctgggcctgggcctgctgaccagcacaggcaacaagtggaggagcaggaggaagatgctgacccccaccttccacttcaccatcctggaggacttcctggacatcatgaatgagcaggccaacatcctggtgaagaagctggagaagcacatcaaccaggaggccttcaactgcttcttctacatcaccctgtgtgccctggacatcatctgtgagacagccatgggcaagaacattggagcccagagcaatgatgacagtgagtatgtgagggctgtgtacaggatgagtgagatgatcttcaggaggatcaagatgccctggctgtggctggacctgtggtacctgatgttcaaggagggctgggagcacaagaagagcctgcagatcctgcacaccttcaccaacagtgtgattgctgagagggccaatgagatgaatgccaatgaggactgcaggggagatggcaggggcagtgcccccagcaagaacaagaggagagccttcctggacctgctgctgagtgtgacagatgatgagggcaacaggctgagccatgaggacatcagggaggaggtggacaccttcatgtttgagggccatgacaccacagctgcagctatcaactggagcctgtacctgctgggcagcaaccctgaggtgcagaagaaggtggaccatgagctggatgatgtgtttggcaagagtgacaggcctgccacagtggaggacctgaagaagctgaggtacctggagtgtgtgatcaaggagaccctgaggctgttccccagtgtgcccctgtttgccaggagtgtgtctgaggactgtgaggtggctggctacagggtgctgaagggcacagaggctgtgatcatcccctatgccctgcacagggaccccaggtacttccccaaccctgaggagttccagcctgagaggttcttccctgagaatgcccagggcaggcacccctatgcctatgtgcccttcagtgctggccccaggaactgcattggccagaagtttgctgtgatggaggagaagaccatcctgagctgcatcctgaggcacttctggattgagagcaaccagaagagggaggagctgggcctggagggccagctgatcctgaggcccagcaatggcatctggatcaagctgaagaggaggaatgctgatgagaggtgagaattcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtttattgcagcttataatggttacaaataaagcaatagcatcacaaatttcacaaataaagcatttttttcactgcattctagttgtggtttgtccaaactcatcaatgtatcttagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 27 - DNA, 4325 bp, synthetic construct
  source 1..4325: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgagacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccatctccagcctcggggctgtccgcagggggacggctgccttcgggggggacggggcagggcggggttcggcttctggcgtgtgaccggcggctctagtgcctctgctaaccatgttcatgccttcttctttttcctacagctcctgggcaacgtgctggttattgtgctgtctcatcattttggcaaaaagcttgccaccatggctggcctgtggctggggctggtctggcagaagctgctcctgtggggggctgcctcagccctctccctggctggggctagcctggtgctgagcctgctgcagagagtggctagctatgctagaaagtggcagcagatgagacccatccccacagtggctagagcctaccccctggtgggccatgccctgctgatgaagcctgatggcagagagttctttcagcagatcattgagtacacagaggagtacagacacatgcccctgctgaagctgtgggtgggccctgtgcctatggtagcactgtacaatgctgagaatgtggaggtgatcctgacaagcagcaagcagattgacaagagcagcatgtacaagttcctggagccctggctgggcctgggcctgctgacaagcactggcaacaagtggagaagcagaagaaagatgctgacccccaccttccacttcaccatcctggaggacttcctggacatcatgaatgagcaagccaacatcctggtgaagaagctggagaagcacatcaaccaagaggccttcaactgcttcttctacatcaccctgtgtgccctggatatcatctgtgagacagccatgggcaagaacattggggctcagagcaatgatgacagtgagtatgtgagagctgtgtacagaatgagtgagatgatcttcagaagaatcaagatgccctggctgtggctggacctgtggtacctgatgttcaaggagggctgggagcacaagaagagcctgcagatcctgcacaccttcaccaactctgtgattgctgagagagccaatgagatgaatgccaatgaggactgcagaggggatggcagaggcagtgcccctagcaagaacaagagaagagccttcctggacctgctcctgtcagtgacagatgatgagggcaacagactgagccatgaggacatcagagaagaggtggacaccttcatgtttgagggccatgacaccacagctgctgccatcaactggagcctgtacctgctgggcagcaaccctgaggtgcagaagaaggtggaccatgagctggatgatgtgtttggcaagagtgacagacctgccacagtggaggacctgaagaagctgagatacctggagtgtgtgatcaaggagaccctgagactgttcccctcagtgcccctgtttgctagaagtgtgagtgaggactgtgaggtggctggctacagagtgctgaagggcacagaggctgtgatcatcccctatgccctgcacagagaccctagatacttccccaaccctgaggagtttcagcctgagagattcttccctgagaatgcccaaggcagacacccctatgcctatgtgcccttcagtgctggccctagaaactgcattgggcagaagtttgctgtgatggaggagaagaccatcctgagctgcatcctgagacacttctggattgagagcaatcagaagagagaggagctgggcctggaggggcagctgatcctgagacctagcaatggcatctggatcaagctgaagagaagaaatgctgatgagagatgagaattcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtttattgcagcttataatggttacaaataaagcaatagcatcacaaatttcacaaataaagcatttttttcactgcattctagttgtggtttgtccaaactcatcaatgtatcttagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 28 - DNA, 4325 bp, synthetic construct
  source 1..4325: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgagacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccatctccagcctcggggctgtccgcagggggacggctgccttcgggggggacggggcagggcggggttcggcttctggcgtgtgaccggcggctctagtgcctctgctaaccatgttcatgccttcttctttttcctacagctcctgggcaacgtgctggttattgtgctgtctcatcattttggcaaaaagcttgccaccatggctggactgtggctgggcctggtgtggcagaagctgctgctgtggggagctgccagtgctctgtctctggctggagcctccctggtgctgagcctgctgcagagggtggcctcctatgccagaaagtggcagcagatgaggcccatccccacagtggccagggcctaccctctggtgggccatgctctgctgatgaagcctgatggcagggagtttttccagcagatcattgagtacacagaggagtacaggcacatgcccctgctgaagctgtgggtgggccctgtgcctatggtggccctgtacaatgctgagaatgtggaggtcattctgacatcctccaagcagattgataagtcctccatgtacaagtttctggagccttggctgggcctgggcctgctgacaagcacaggcaataagtggaggagcaggagaaagatgctgacccctacctttcactttacaatcctggaggactttctggatatcatgaatgagcaggccaacatcctggtgaagaagctggagaagcacatcaaccaggaggccttcaattgtttcttctacatcacactgtgtgccctggacatcatctgtgagacagccatgggcaagaatattggagcccagtccaatgatgattctgagtatgtgagggctgtgtacaggatgagtgagatgatctttaggagaatcaagatgccctggctgtggctggacctgtggtacctgatgtttaaggagggctgggagcacaagaagagcctgcagatcctgcacacattcacaaattctgtgattgctgagagagccaatgagatgaatgccaatgaggactgtagaggagatggcaggggctctgccccctctaagaataagaggagggccttcctggacctgctgctgtctgtgacagatgatgagggcaataggctgtcccatgaggatatcagggaggaggtggacaccttcatgtttgagggccatgacacaacagctgctgccatcaactggagcctgtacctgctgggcagcaatcctgaggtgcagaagaaggtggatcatgagctggatgatgtgtttggcaagagtgacagacctgccacagtggaggatctgaagaagctgagatacctggagtgtgtgatcaaggagacactgagactgttcccttctgtgcccctgtttgccagaagtgtgtctgaggactgtgaggtggctggctacagagtgctgaagggcacagaggctgtgatcatcccctatgccctgcacagggatcctagatacttccctaaccctgaggagttccagcctgagaggttcttccctgagaatgcccagggcaggcacccctatgcctatgtgcccttcagtgctggccctaggaactgtattggccagaagtttgctgtgatggaggagaagaccatcctgtcctgcatcctgagacacttctggattgagtccaaccagaagagggaggagctgggcctggagggccagctgatcctgagacctagcaatggcatctggatcaagctgaagagaagaaatgctgatgagagatgagaattcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtttattgcagcttataatggttacaaataaagcaatagcatcacaaatttcacaaataaagcatttttttcactgcattctagttgtggtttgtccaaactcatcaatgtatcttagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 29 - DNA, 4325 bp, synthetic construct
  source 1..4325: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgagacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccatctccagcctcggggctgtccgcagggggacggctgccttcgggggggacggggcagggcggggttcggcttctggcgtgtgaccggcggctctagtgcctctgctaaccatgttcatgccttcttctttttcctacagctcctgggcaacgtgctggttattgtgctgtctcatcattttggcaaaaagcttgccaccatggcaggactgtggctgggactggtgtggcagaagctgctgctgtggggagcagcaagtgccctgtccctggcaggagcctccctggtgctgtctctgctgcagagggtggcaagctatgcaaggaagtggcagcagatgaggcccatccctacagtggcaagagcatatccactggtgggacatgcactgctgatgaagcctgatggcagggagttctttcagcagatcattgagtacacagaggagtataggcacatgccactgctgaagctgtgggtgggaccagtgccaatggtggccctgtacaatgctgagaatgtggaagtgatcctgaccagctccaagcagattgataagtctagcatgtataagttcctggagccttggctgggactgggactgctgacctccacaggcaacaagtggaggtctaggagaaagatgctgaccccaacattccactttacaatcctggaggacttcctggatatcatgaatgagcaggccaatatcctggtgaagaagctggagaagcacatcaaccaggaggcctttaattgcttcttctacatcaccctgtgtgccctggacatcatctgtgagacagccatgggcaagaacattggagcccagagcaatgatgattctgagtatgtgagggctgtgtataggatgtctgagatgatctttaggaggatcaagatgccctggctgtggctggatctgtggtatctgatgttcaaggagggctgggagcacaagaagagcctgcagatcctgcacaccttcacaaactctgtgattgctgagagggccaatgagatgaatgccaatgaggactgtaggggagatggaagaggctctgcccctagcaagaacaagaggagagccttcctggacctgctgctgtctgtgacagatgatgagggcaatagactgagccatgaggacatcagggaggaggtggatacattcatgtttgagggacatgacaccacagcagcagccatcaactggtctctgtacctgctgggcagcaatccagaggtgcagaagaaggtggatcatgagctggatgatgtgtttggcaagagtgacagacctgccacagtggaggatctgaagaagctgaggtacctggagtgtgtgatcaaggagacactgagactgttcccctctgtgcctctgtttgccaggtctgtgtctgaggactgtgaggtggctggctatagggtgctgaagggcacagaggctgtgatcatcccttatgccctgcacagggaccccagatatttccctaacccagaggagtttcagccagagaggttcttcccagagaatgcacagggcaggcacccttatgcctatgtgccattctctgctggaccaaggaactgcattggacagaagtttgctgtgatggaggagaagaccatcctgtcctgtatcctgaggcacttctggattgagtctaatcagaagagggaggagctgggactggagggacagctgatcctgagaccctccaatggcatctggatcaagctgaagaggaggaatgctgatgagaggtgagaattcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtttattgcagcttataatggttacaaataaagcaatagcatcacaaatttcacaaataaagcatttttttcactgcattctagttgtggtttgtccaaactcatcaatgtatcttagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 30 - DNA, 4325 bp, synthetic construct
  source 1..4325: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgagacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccatctccagcctcggggctgtccgcagggggacggctgccttcgggggggacggggcagggcggggttcggcttctggcgtgtgaccggcggctctagtgcctctgctaaccatgttcatgccttcttctttttcctacagctcctgggcaacgtgctggttattgtgctgtctcatcattttggcaaaaagcttgccaccatggctgggctgtggctggggctggtgtggcagaagctgctgctgtggggagctgctagtgccctcagcctggctggagcaagcctggtgctgagtctcctgcagagagtggcctcctatgccaggaagtggcaacagatgaggcccatcccaacagtggctagagcatacccactggtgggccatgccttgctgatgaagcctgatggcagggagttctttcagcagatcattgaatacactgaggagtacagacacatgcctctgctgaagctgtgggtgggcccagtgcctatggtggccctctacaatgctgagaatgtggaagtgattctgaccagtagcaagcagattgataagagtagcatgtacaagttcctggagccctggctggggttaggcctgctgaccagcacaggaaacaagtggaggagtaggaggaagatgctgacccccaccttccacttcaccatcctggaggactttctggatattatgaatgaacaggcaaacatcttggtgaagaagctggagaagcacattaatcaggaggccttcaactgcttcttctatattaccctgtgtgccctggatatcatctgtgagacagccatgggaaagaatattggagcccagagtaatgatgactctgagtatgtgagggctgtgtacagaatgagtgagatgatcttcaggagaatcaagatgccatggctgtggctggacctgtggtacctgatgttcaaggaggggtgggagcacaagaagtccctgcagattctgcacacctttaccaacagtgtgattgctgagagggccaatgagatgaatgccaatgaggattgcaggggagatggaagaggctctgcccccagcaagaacaaaagaagggccttcctggacctgctgttgtctgtgactgatgatgaagggaacaggctgtcacatgaggacatcagagaggaggtggacaccttcatgtttgagggccatgacaccacagctgctgccattaactggagcctgtacctgctgggaagcaacccagaggtgcagaagaaggtggaccatgagctggatgatgtgtttggcaagtcagacaggcctgccacagtggaggacctgaagaagctgaggtacctggagtgtgtgatcaaggaaacactgaggctgttcccaagtgtgcctctgtttgccaggtctgtgtcagaggactgtgaagtggctggctacagggtgctgaagggaacagaggcagtgatcatcccatatgcactgcacagggaccctaggtattttcctaaccctgaggagttccagccagagaggttctttcctgagaatgctcagggcaggcatccttatgcctatgtgcctttctctgctggccccaggaactgcattggccagaagtttgctgtgatggaagagaagaccattttgtcctgtattctgaggcacttctggattgaatccaaccagaagagggaggagctgggcctggaagggcagctgatcctgaggccctccaatggcatttggatcaaactgaagaggagaaatgctgatgagagatgagaattcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtttattgcagcttataatggttacaaataaagcaatagcatcacaaatttcacaaataaagcatttttttcactgcattctagttgtggtttgtccaaactcatcaatgtatcttagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 31 - DNA, 4325 bp, synthetic construct
  source 1..4325: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgagacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccatctccagcctcggggctgtccgcagggggacggctgccttcgggggggacggggcagggcggggttcggcttctggcgtgtgaccggcggctctagtgcctctgctaaccatgttcatgccttcttctttttcctacagctcctgggcaacgtgctggttattgtgctgtctcatcattttggcaaaaagcttgccaccatggctggcctgtggctgggcctggtgtggcagaagctgctcctgtggggagctgcctctgccctgtccctggctggggctagcctggtgctgtctctgttgcagagagtggccagctatgccaggaagtggcaacagatgagacccatccccacagtggccagagcataccccctggtgggccatgccctgctgatgaagcctgatggcagagagttcttccagcagatcattgagtacacagaggaatacagacacatgcctctgctgaagctgtgggtgggccctgtgcctatggtggctctctacaatgctgagaatgtggaagtgatcctgaccagcagcaagcagattgacaagtctagcatgtacaagttcctggagccttggctgggactgggactgctgaccagcacaggcaacaagtggaggagcagaagaaagatgctgacacctaccttccactttaccatcctggaagatttcctggacattatgaatgagcaggccaacatcctggtcaagaagctggaaaagcacatcaaccaggaggcttttaactgcttcttctacatcaccctgtgtgccctggacatcatctgtgagacagccatgggcaagaacattggagctcagagcaatgatgatagtgaatatgttagagctgtctacaggatgagtgagatgatcttcaggagaatcaagatgccctggctgtggctggatctgtggtacctgatgttcaaggaaggatgggagcacaagaaaagcctgcagatcctgcacaccttcaccaacagtgtgattgctgagagagccaatgagatgaatgccaatgaggactgtagaggagatggcaggggcagtgccccttctaagaacaagagaagggccttcctggacctgctgctgagtgttacagatgatgagggcaatagactgtctcatgaggatatcagagaagaggtggacaccttcatgtttgagggccatgacactacagctgctgctattaactggtccctgtacctgctgggcagcaaccctgaagtgcagaagaaggtggaccatgagctggatgatgtgtttggaaagagtgatagacctgccacagtggaagacctgaagaagctaaggtacctggagtgtgtgatcaaggaaacactgagactgttcccctctgtgcctctgtttgccagaagtgtgtctgaggattgtgaggtggctggctacagggtgctgaagggcacagaggctgtgatcatcccttatgccctgcacagggaccccagatatttccctaaccctgaggagttccagcctgagaggttcttcccagagaatgcccagggcagacatccttatgcctatgtgccattcagtgctggtcctagaaactgcattggccagaagtttgctgtgatggaagagaagaccatcctgagctgcatcctgaggcacttctggattgagtctaatcagaagagagaggaactgggcctggaaggccagctgatcctcaggccaagcaatggcatctggatcaagctgaagaggaggaatgctgatgagaggtgagaattcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtttattgcagcttataatggttacaaataaagcaatagcatcacaaatttcacaaataaagcatttttttcactgcattctagttgtggtttgtccaaactcatcaatgtatcttagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 32 - DNA, 4325 bp, synthetic construct
  source 1..4325: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgagacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccatctccagcctcggggctgtccgcagggggacggctgccttcgggggggacggggcagggcggggttcggcttctggcgtgtgaccggcggctctagtgcctctgctaaccatgttcatgccttcttctttttcctacagctcctgggcaacgtgctggttattgtgctgtctcatcattttggcaaaaagcttgccaccatggctggactgtggctgggactggtgtggcagaaactgctgctttggggagctgcctctgctctgtctcttgctggtgcttctctggtgctgagcctgctgcagagagtggcctcttatgccagaaagtggcagcagatgaggcccattcctacagtggccagagcctatcctcttgtgggacatgccctgctgatgaagcctgatggcagagagttcttccagcagatcattgagtacacagaggaatacaggcacatgcccctgctcaagctgtgggttggacctgtgcctatggtggccctgtacaatgctgagaatgtggaagtgatcctgaccagcagcaagcagattgacaagtccagcatgtacaagtttctggaaccctggctgggcctgggcctgctgacatctactggaaacaagtggaggagcaggaggaagatgctgacccctaccttccactttacaattctggaggacttcctggacatcatgaatgagcaggccaacatcctggtcaagaagctggaaaagcacatcaatcaagaggccttcaactgcttcttctacatcaccctgtgtgccctggatatcatctgtgagacagccatgggcaagaacattggagcccagagcaatgatgatagtgaatatgtgagggctgtgtacaggatgagtgagatgatcttcaggaggatcaagatgccttggctgtggctggacctgtggtatctgatgttcaaagaaggctgggagcacaagaagtccctgcagatcctgcacaccttcaccaacagtgtgattgctgagagggccaatgagatgaatgccaatgaagattgcagaggagatggcaggggaagtgcccctagcaagaacaagagaagggccttcctggatctgctgctgagtgtgacagatgatgagggcaatagactgagccatgaggacatcagagaagaggtggacaccttcatgtttgagggccatgatacaacagctgctgccatcaattggagcctgtacctgctgggcagcaaccctgaggtgcagaagaaggtggaccatgagctggatgatgtgtttggcaagtctgatagacctgccacagtggaagatctgaagaagctgagatacctggaatgtgtgatcaaagagacactgaggctgttccctagtgtgcccctgtttgccagaagtgtgtctgaggattgtgaagtggctggctacagagtgctgaagggaacagaggctgtgatcatcccctatgctctgcacagagatcccaggtacttccccaatcctgaagagttccagcctgagaggttcttcccagagaatgcccagggcagacacccctatgcctatgtgccattctctgctggacctaggaactgcattggccagaagtttgctgtgatggaagagaaaaccatcctgagctgtatcctgaggcacttctggattgagagcaaccagaagagagaggaactgggcctggagggacagctgattctgaggcctagcaatggcatctggatcaagctgaagaggagaaatgctgatgagaggtgagaattcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtttattgcagcttataatggttacaaataaagcaatagcatcacaaatttcacaaataaagcatttttttcactgcattctagttgtggtttgtccaaactcatcaatgtatcttagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 33 - DNA, 4325 bp, synthetic construct
  source 1..4325: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgagacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccatctccagcctcggggctgtccgcagggggacggctgccttcgggggggacggggcagggcggggttcggcttctggcgtgtgaccggcggctctagtgcctctgctaaccatgttcatgccttcttctttttcctacagctcctgggcaacgtgctggttattgtgctgtctcatcattttggcaaaaagcttgccaccatggctggactttggctgggactggtgtggcagaagctgctgctgtggggagctgctagtgccctgagcctggctggagcttctcttgtgctgagcttactgcaaagagtggccagctatgccagaaagtggcagcagatgagacccatccccacagtggctagagcttatcctctggtgggacatgctctgctgatgaagcctgatggaagagagtttttccagcagatcattgagtacacagaggagtacagacacatgcccctgctgaagctgtgggtgggcccagttcctatggtggctctgtataatgctgagaatgtggaggtgatcctgaccagcagcaagcagattgacaagagcagcatgtacaagttcctggagccctggctgggcctgggattactgacatctacaggaaataaatggagaagcagaagaaagatgctgacccccaccttccacttcaccatcctggaggacttcctggacatcatgaatgagcaggccaacatcctggtgaagaagctggagaagcacatcaaccaggaggccttcaactgcttcttctacatcaccctgtgtgccctggacatcatctgtgagacagctatgggcaagaacattggagctcagagcaatgatgacagtgagtatgtgagagctgtgtacagaatgagtgagatgatcttcagaagaatcaagatgccctggctgtggctggacctgtggtatctgatgttcaaggagggctgggagcacaagaagagcctgcagatcctgcacaccttcaccaacagtgtgattgctgagagagccaatgagatgaatgccaatgaggactgcagaggagatggcagaggatctgctcctagcaaaaataagagaagagccttcctggacctgctgctgagtgtgacagatgatgaaggaaacagactgagccatgaggacatcagagaggaggtggacaccttcatgtttgagggccatgacaccacagctgctgctattaattggagcctgtatctgctgggaagcaaccctgaagtgcagaagaaggtggaccatgagctggatgatgtgtttggcaagagtgacagacctgccacagtggaggatctgaagaagctgagatacctggagtgtgtgatcaaggagaccctgagactgttccccagtgtgcccctgtttgctagatctgtgagtgaagattgtgaggtggctggctatagagtgctgaaaggcacagaagctgtgatcatcccctatgctctgcacagagaccccagatacttccccaaccctgaggagtttcagcctgagagattcttccctgagaatgcccaaggcagacacccctatgcttatgtgcccttcagtgctggccccagaaattgcattggacagaagtttgctgtgatggaggagaagaccatcctgagctgcatcctgagacacttctggattgagagcaaccagaagagagaggagctgggcctggagggccaactgattctgagacctagcaatggcatctggatcaagctgaagagaaggaatgctgatgagagatgagaattcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtttattgcagcttataatggttacaaataaagcaatagcatcacaaatttcacaaataaagcatttttttcactgcattctagttgtggtttgtccaaactcatcaatgtatcttagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 34 - DNA, 4325 bp, synthetic construct
  source 1..4325: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgagacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccatctccagcctcggggctgtccgcagggggacggctgccttcgggggggacggggcagggcggggttcggcttctggcgtgtgaccggcggctctagtgcctctgctaaccatgttcatgccttcttctttttcctacagctcctgggcaacgtgctggttattgtgctgtctcatcattttggcaaaaagcttgccaccatggctggactgtggctgggactggtgtggcagaaactgctgctgtggggagctgcttctgcactgtctctggctggggcatctctggtcctgtccctgctgcagagagtggcctcctatgccagaaagtggcagcagatgagacctatccccacagtggccagggcataccccctggtgggtcatgccctcctgatgaaaccagatgggagggaatttttccagcagattattgagtacacagaagaatatagacacatgcccctcctcaagctgtgggttgggcctgtccctatggttgccctctataatgctgagaatgttgaagttattctcacatcatctaagcagattgataagagtagcatgtataagtttctggaaccctggctggggctggggctcctcacctcaactgggaacaagtggaggtctagaagaaaaatgctcacccccaccttccatttcaccatcctggaggactttctggacatcatgaatgagcaggctaacatcctggttaaaaaactggagaaacatatcaatcaggaagccttcaattgtttcttctatattactctctgtgctctggacattatctgtgagactgcaatgggaaagaatattggtgcacagtcaaatgatgattcagaatatgtcagggctgtctacagaatgtctgagatgatctttaggagaatcaagatgccctggctgtggctggatctgtggtacctcatgttcaaggaaggatgggagcataagaaaagtctgcagattctgcataccttcaccaactcagtgattgcagagagagcaaatgagatgaatgctaatgaagattgcaggggggatggaaggggaagtgcccctagcaaaaataagagaagggcctttctggatctcctcctgagtgttacagatgatgagggtaatagactcagccatgaagatatcagagaagaggtggacacattcatgtttgagggccatgatactactgcagcagctattaactggagtctgtacctgctgggctcaaaccctgaggttcagaagaaagttgaccatgaactggatgatgtctttgggaagtcagacaggcctgctactgtggaagatctgaagaagctgaggtacctggagtgcgtcattaaggaaaccctgagactcttcccctctgtccccctgtttgctagatcagtttcagaagattgtgaggtggctggctatagggtcctgaagggtactgaggcagtgattatcccttatgctctccacagggatcctaggtactttccaaatcctgaggagttccagcctgagagatttttcccagaaaatgcccagggcaggcatccctatgcttatgtcccctttagtgcaggaccaagaaactgtattggccagaaatttgcagtcatggaggagaaaacaatcctgtcttgcatcctgaggcatttctggattgaatccaaccagaaaagagaggagctgggactggaagggcagctgattctgagaccaagcaatgggatttggatcaagctgaagaggaggaatgcagatgagaggtaagaattcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtttattgcagcttataatggttacaaataaagcaatagcatcacaaatttcacaaataaagcatttttttcactgcattctagttgtggtttgtccaaactcatcaatgtatcttagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 35 - DNA, 1671 bp, synthetic construct
  source 1..1671: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccatctccagcctcggggctgtccgcagggggacggctgccttcgggggggacggggcagggcggggttcggcttctggcgtgtgaccggcggctctagtgcctctgctaaccatgttcatgccttcttctttttcctacag

SEQ ID NO: 36 - DNA, 2796 bp, synthetic construct
  source 1..2796: mol_type = other DNA
ctgcgcgctcgctcgctcactgaggccgcccgggcaaagcccgggcgtcgggcgacctttggtcgcccggcctcagtgagcgagcgagcgcgcagagagggagtggggttaaacgttgacattgattattgcggcctctagactcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcaaagcttgccgccaccatggcggggctctggctggggctcgtgtggcagaagctgctgctgtggggcgcggcgagtgccctttccctggccggcgccagtctggtcctgagcctgctgcagagggtggcgagctacgcgcggaaatggcagcagatgcggcccatccccacggtggcccgcgcctacccactggtgggccacgcgctgctgatgaagccggacgggcgagaattttttcagcagatcattgagtacacagaggaataccgccacatgccgctgctgaagctctgggtcgggccagtgcccatggtggccctttataatgcagaaaatgtggaggtaattttaactagttcaaagcaaattgacaaatcctctatgtacaagtttttagaaccatggcttggcctaggacttcttacaagtactggaaacaaatggcgctccaggagaaagatgttaacacccactttccattttaccattctggaagatttcttagatatcatgaatgaacaagcaaatatattggttaagaaacttgaaaaacacattaaccaagaagcatttaactgctttttttacatcactctttgtgccttagatatcatctgtgaaacagctatggggaagaatattggtgctcaaagtaatgatgattccgagtatgtccgtgcagtttatagaatgagtgagatgatatttcgaagaataaagatgccctggctttggcttgatctctggtaccttatgtttaaagaaggatgggaacacaaaaagagccttcagatcctacatacttttaccaacagtgtcatcgctgaacgggccaatgaaatgaacgccaatgaagactgtagaggtgatggcaggggctctgccccctccaaaaataaacgcagggcctttcttgacttgcttttaagtgtgactgatgacgaagggaacaggctaagtcatgaagatattcgagaagaagttgacaccttcatgtttgaggggcacgatacaactgcagctgcaataaactggtccttatacctgttgggttctaacccagaagtccagaaaaaagtggatcatgaattggatgacgtgtttgggaagtctgaccgtcccgctacagtagaagacctgaagaaacttcggtatctggaatgtgttattaaggagacccttcgcctttttccttctgttcctttatttgcccgtagtgttagtgaagattgtgaagtggcaggttacagagttctaaaaggcactgaagccgtcatcattccctatgcattgcacagagatccgagatacttccccaaccccgaggagttccagcctgagcggttcttccccgagaatgcacaagggcgccatccatatgcctacgtgcccttctctgctggccccaggaactgtataggtcaaaagtttgctgtgatggaagaaaagaccattctttcgtgcatcctgaggcacttttggatagaatccaaccagaaaagagaagagcttggtctagaaggacagttgattcttcgtccaagtaatggcatctggatcaagttgaagaggagaaatgcagatgaacgccatatgtacccatacgatgttccagattacgctagcggcttcgccaacgagcttggacccaggttgatgggaaagtaagcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcgttactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccag